(19) United States
(12) Patent Application Publication
(10) Pub. No.: US 2026/0091100 A1
(43) Pub. Date: Apr. 2, 2026

(54) NADC34-LIKE PRRSV-2 VACCINE CANDIDATE STRAIN AND APPLICATION THEREOF

(71) Applicant: YANGZHOU UNIVERSITY, Jiangsu (CN)

(72) Inventors: Nanhua Chen, Jiangsu (CN); Ming Qiu, Jiangsu (CN); Hong Lin, Jiangsu (CN); Jianzhong Zhu, Jiangsu (CN)

(73) Assignee: YANGZHOU UNIVERSITY, Jiangsu (CN)

(21) Appl. No.: 19/256,185

(22) Filed: Jul. 1, 2025

(30) Foreign Application Priority Data
Sep. 27, 2024 (CN) 202411356016.6

(51) Int. Cl. A61K 39/12; A61K 39/00; A61P 37/04; C12N 7/00; C12N 15/86

(52) U.S. Cl. CPC A61K 39/12; A61P 37/04; C12N 7/00; C12N 15/86; A61K 2039/5252; C12N 2770/10021; C12N 2770/10034; C12N 2770/10043; C12N 2770/10051

(57) ABSTRACT

Disclosed are an rBJ-VVL plasmid, a mutant strain of NADC34-like PRRSV-2 and a preparation method therefor and application thereof. Further disclosed is an NADC34-like PRRSV-2-specific vaccine. In the present disclosure, a modified strain rBJ-VVL with tropism for Marc-145 cells is obtained by precisely mutating an amino acid at positions 91/97/98 of GP2a; the modified virus constructed in the present disclosure can be propagated in Marc-145 cells, cause cytopathic effects and form plaques when inoculated into Marc-145 cells for serial passage; the resulting Marc-145 cell-passaged viruses have an extremely viral load, and a large number of new progeny viruses can be obtained in a short time; and the Marc-145-adapative modified strain cultured in the present disclosure is used to create a first NADC34-like PRRSV-2-specific vaccine.

### CROSS-REFERENCE TO RELATED APPLICATION

This application claims the priority benefit of China application serial no. 202411356016.6, filed on Sep. 27, 2024. The entirety of each of the above-mentioned patent applications is hereby incorporated by reference herein and made a part of this specification.

### REFERENCE TO A SEQUENCE LISTING

The instant application contains a Sequencing Listing which has been submitted electronically in XML file and is hereby incorporated by reference in its entirety. Said XML copy, created on Jun. 10, 2025, is named 156861-US-Sequence List and is 55,930 bytes in size.

### TECHNICAL FIELD

The present disclosure belongs to the technical field of veterinary biological products, and particularly relates to an NADC34-like PRRSV-2 vaccine candidate strain and application thereof.

### RELATED ART

Porcine reproductive and respiratory syndrome virus (PRRSV) poses a severe threat to the pig farming sector around the globe, resulting in significant economic losses. PRRSV infection can lead to abortions in sows and respiratory syndrome in piglets. The latest research divides PRRSV into PRRSV-1 and PRRSV-2. In China, PRRSV-2 strains are predominant, with highly pathogenic PRRSV-2 (HP-PRRSV-2), NADC30-like PRRSV-2, and NADC34-like PRRSV-2 being the main ones. At present, NADC34-like PRRSV-2 is one of the most widespread strains and has caused significant economic damage to the pig industry. Therefore, there is an urgent need for specific vaccines to prevent and control the epidemic.

PRRSV-2 is a positive-sense single-stranded RNA virus belonging to the family Arteriviridae. Its genome is approximately 15 kilobases (kb) in length and contains 10 open reading frames (ORFs). ORF1a and ORF1b encode at least 16 nonstructural proteins (Nsps), and ORF2-7 encodes eight structural proteins. Among them, the ORF2-ORF4 genes encode three minor envelope glycoproteins (GP2a, GP3, and GP4), which form a heterotrimer on the viral envelope. Previous reports have stated that GP2a-GP3-GP4 determine the cell tropism of arterivirus.

Vaccination remains an important measure for epidemic control in China. However, there is no report on a specific vaccine strain for the NADC34-like PRRSV-2 yet. In addition, wild-type NADC34-like PRRSV-2 strains are unable to adapt to in vitro culture of Marc-145 cells, which has become a major obstacle to the development of strain-specific vaccines.

In the previous study, an infectious clone virus of an NADC34-like PRRSV-2 rBJ1805-2 strain (Invention title: Construction and Application of Reverse Genetics Platform for NADC34-like Porcine Reproductive and Respiratory Syndrome Virus; Chinese Patent Publication No.: CN115992100A) has been established. This infectious clone is deposited with the China Center for Type Culture Collection (CCTCC), with a deposit address being Wuhan University, Wuhan, China, a deposit number being CCTCC NO.: V202250 and a deposit date being Jun. 29, 2022. In the aforesaid previous study, the ORF2-4 of the rBJ1805-2 infectious clone was entirely replaced with an ORF2-4CON, to enable it to adapt to Marc-145 cell culture. However, the key amino acid sites in ORF2-4 that enables the wild-type NADC34-like PRRSV-2 strain to adapt to Marc-145 cells have not yet been accurately analyzed, and there have been no reports on the development of vaccine specific to NADC34-like PRRSV-2 strains.

### SUMMARY OF INVENTION

Objectives of the present disclosure: one technical problem to be solved by the present disclosure aims to provide an rBJ-VVL plasmid capable of transfecting cells to obtain a viral strain and a method for constructing the same.

In view of the NADC34-like PRRSV-2 that has appeared and become widely prevalent in China in recent years, another technical problem to be solved by the present disclosure is to provide a mutant strain of NADC34-like PRRSV-2, a passaged strain thereof, or a method for obtaining the same. Immunization and challenge test in piglets using the passaged virus have demonstrated that the modified strain had a complete clinical protective effect against the wild-type NADC34-like PRRSV-2 strain.

The final technical problem to be solved by the present disclosure is to provide an NADC34-like PRRSV-2-specific vaccine.

Technical solution: the present disclosure provides an rBJ-VVL plasmid. The rBJ-VVL plasmid is obtained by using a plasmid of a reverse genetics platform pACYC177-rBJ1805-2 as a template, designing a primer pair for amino acid mutations at positions 91, 97, and 98 of GP2a protein of the rBJ1805-2 virus to V, V and L, respectively, amplifying to obtain a mutated fragment by a PCR amplification method and then ligating the mutated fragment with a linearized vector.

Specifically, the primer pair used includes a combination of rBJ-XbaI-F3 and rBJ-VVL-R, and a combination of rBJ-VVL-F and rBJ-NOT1-2fu-1, which are amplified by the PCR amplification method to obtain front and rear segments of an rBJ-VVL mutant, respectively, and the rBJ-VVL plasmid is obtained by homologous recombination of the front and rear segments with the linearized vector. Sequences of the primer pair are shown in the table below:

| | | | Length |
|---|---|---|---|
| Primer name | Sequence (5′-3′) | SEQ ID NO: | (bp) |
| rBJ-VVL-F | CGTCTGGGGAgtCAAGCACCCCCTGGGAgT | SEQ ID NO: 3 | 46 |
| | GcTTTGGCACCACAAG | | |
| rBJ-VVL-R | CTTGTGGTGCCAAAgCAcTCCCAGGGGGTG | SEQ ID NO: 4 | 46 |
| | CTTGacTCCCCAGACG | | |
| rBJ-XbaI-F3 | CGCTGCAATACTCATGGATAGTTGTGCTTG | SEQ ID NO: 5 | 31 |
| | T | | |
| rBJ-NOT1- | CAAACAACAGATGGCTGGCAACTAGAAGG | SEQ ID NO: 6 | 34 |
| 2fu-1 | CACAG | | |

The present disclosure further provides a mutant strain of NADC34-like PRRSV-2, where the mutant strain is obtained by mutating amino acids at positions 91, 97, and 98 of the GP2a protein of an isolate BJ1805-2 of the NADC34-like PRRSV-2 from T, M and F to V, V and L, respectively.

The mutant strain is obtained by transfecting cells with the mutant plasmid.

In one aspect, the present disclosure provides a precise amino acid modification method for mutating the amino acids at positions 91/97/98 of the GP2a protein of the isolate BJ1805-2 of the NADC34-like PRRSV-2 from a T/M/F pattern to a V/V/L pattern, thereby making the same adaptive to Marc-145 cell culture. Specifically, based on a reverse genetics platform for the NADC34-like PRRSV-2, a large number of chimeric viruses were used to perform the construction and screening, amino acids at positions 91, 97, and 98 of NADC34-like PRRSV-2 GP2a were mutated into the V/V/L pattern, making it adaptive to the serial passage in Marc-145 cells. The Marc-145 cell-adaptive strain rBJ-VVL was obtained. In addition, the modified viruses rBJ-TVL, rBJ-VML and rBJ-VVF that were reverted to mutations at three points in the V/V/L pattern were all unable to adapt to Marc-145 culture, proving that the three point mutation pattern is the most accurate mutation pattern for conferring Marc-145 cell tropism to NADC34-like PRRSV-2.

The present disclosure further provides a method for constructing an rBJ-VVL plasmid, including the following steps:
(1) designing a mutant primer pair targeting amino acids at positions 91, 97, and 98 of the GP2a protein of the rBJ1805-2 virus, and the sequences of the primer pairs are shown in the table below:

| | | | Length |
|---|---|---|---|
| Primer name | Sequence (5′-3′) | SEQ ID NO: | (bp) |
| rBJ-VVL-F | CGTCTGGGGAgtCAAGCACCCCCTGGGAgT | SEQ ID NO: 3 | 46 |
| | GcTTTGGCACCACAAG | | |
| rBJ-VVL-R | CTTGTGGTGCCAAAgCAcTCCCAGGGGGTG | SEQ ID NO: 4 | 46 |
| | CTTGacTCCCCAGACG | | |
| rBJ-XbaI-F3 | CGCTGCAATACTCATGGATAGTTGTGCTTG | SEQ ID NO: 5 | 31 |
| | T | | |
| rBJ-NOT1- | CAAACAACAGATGGCTGGCAACTAGAAGG | SEQ ID NO: 6 | 34 |
| 2fu-1 | CACAG | | |

(2) using a plasmid of a reverse genetics platform pACYC177-rBJ1805-2 as a template, performing PCR amplification by using a combination of rBJ-XbaI-F3 and rBJ-VVL-R, and a combination of rBJ-VVL-F and rBJ-NOT1-2fu-1 to obtain front and rear segments of an rBJ-VVL mutant, and performing homologous recombination of the linearized vector fragment, the front fragment, and the rear fragment to obtain the rBJ-VVL plasmid.

The present disclosure further provides a method for obtaining the mutant strain or the passaged strain of NADC34-like PRRSV-2, where the method includes the above-mentioned steps (1) and (2), and further includes a step (3) of transfecting cells with the rBJ-VVL plasmid; or performing further serial passage to obtain the passaged strain.

The cells include PAM cells or Marc-145 cells.

In another aspect, the present disclosure provides an in vitro passaged virus strain of NADC34-like PRRSV-2 highly adaptive to Marc-145 cells. Specifically, the rBJ-VVL virus is subjected to continuous in vitro serial passage in Marc-145 cells, the TCID₅₀ growth curves in Marc-145 cells are determined for the virus strains passaged 10, 30, and 50 times, respectively, and an NADC34-like PRRSV-2 virus strain with extremely high yield is obtained.

The rBJ-VVL plasmid, the virus strain, and its passaged strains of the present disclosure may be used in the preparation of a vaccine for preventing a porcine reproductive and respiratory syndrome virus.

The present disclosure further provides an NADC34-like PRRSV-2-specific vaccine, the NADC34-like PRRSV-2-specific vaccine includes the virus strain, a passaged strain thereof, or an inactivated strain thereof.

The vaccine is an injectable formulation, a drop formulation, a spray formulation, or other dosage forms.

In a last aspect, the present disclosure provides evaluation results of the immune protection effect of the parental strain rBJ1805-2, which is unable to adapt to Marc-145 cells, as well as of the 10^{th} and 30^{th} generation in vitro passaged strains of rBJ-VVL. Specifically, piglets are immunized with the rBJ1805-2 strain, 10^{th} and 30^{th}-passage rBJ-VVL strains, and a challenge with a wild-type NADC34-like PRRSV-2 strain is conducted 42 days after immunization. During the period, body temperature, body weight, and viremia of the piglets are monitored. After the challenge, lung lesions of the piglets are examined, and HE staining and immunohistochemistry are performed. The results indicate that passaged strains derived from rBJ-VVL are not only safe but also capable of providing complete clinical protection in pigs against wild-type NADC34-like PRRSV-2 challenge. Therefore, the strain can be used to develop a first NADC34-like PRRSV-2-specific vaccine.

Beneficial effects: Compared with the prior art, the present disclosure has the following benefits: based on the reverse genetics platform for the NADC34-like PRRSV-2, the present disclosure obtains a modified strain rBJ-VVL with tropism for Marc-145 cells by precisely mutating the amino acid at positions 91/97/98 of GP2a; the modified virus constructed in the present disclosure can be propagated in Marc-145 cells can cause cytopathic effects (CPE) and form plaques when inoculated into Marc-145 cells for serial passage; the 1^{st}-generation adaptive virus of rBJ-VVL is used as the parental strain, and the rBJ-VVL-P10, rBJ-VVL-P30, and rBJ-VVL-P50 strains of NADC34-like PRRSV-2 adaptive to Marc-145 cells are cultured through continuous in vitro passage of Marc-145 cells; the resulting Marc-145 cell-passaged viruses obtained in the present disclosure have an extremely viral load, and a large number of new progeny viruses can be obtained in a short time (reaching 10^{6.9} TCID₅₀/mL); the Marc-145 cell-passaged viruses obtained in the present disclosure exhibit excellent safety, neither inducing fever (in vitro <40° C.) after inoculation into piglets, nor causing piglet mortality; when challenged with the wild-type strain NADC34-like PRRSV-2, the passaged viruses of the Marc-145 cells provide complete clinical protection; and the Marc-145-adaptive modified virus strain cultured in the present disclosure is used to create a first NADC34-like PRRSV-2-specific vaccine, which is conducive to effective prevention and control of PRRS epidemic in China.

### BRIEF DESCRIPTION OF DRAWINGS

FIG. 1 is a schematic diagram illustrating a strategy for screening key structural proteins of Marc-145 cell tropism of an infectious clone virus rBJ1805-2 according to Example 1.

FIG. 2 shows indirect immunofluorescence images of PAM cells and Marc-145 cells rescued with various modified strains according to Example 1.

FIG. 3A and FIG. 3B show growth curves and plaque images of Marc-145 cells in various modified strains according to Example 1.

FIG. 4 is a schematic diagram of comparison of rBJ-VVL and positions 91/97/98 with other strains according to Example 1.

FIG. 5 shows indirect immunofluorescence images of PAM cells and Marc-145 cells infected with rBJ-VVL and single-site reverse mutant strains at positions 91/97/98 according to Example 1.

FIG. 6 shows TCID₅₀ growth curves of Marc-145 cells of 10^{th}, 30^{th}, and 50^{th} passages of rBJ-VVL according to Example 1.

FIG. 7 shows plaques formed in Marc-145 cells at 10^{th}, 30^{th}, and 50^{th} passages of rBJ-VVL according to Example 1.

FIG. 8 shows viremia produced in piglets after immunization and challenge with BJ1805-2 and rBJ-VVL strains at 10^{th} and 30^{th} passages according to Example 2.

FIG. 9 shows dynamic changes in body temperature of piglets inoculated during immunization and challenge with BJ1805-2 and rBJ-VVL strains of 10^{th} and 30^{th} passages according to Example 2.

FIG. 10 shows body weight changes of piglets inoculated during immunization and challenge with BJ1805-2 and rBJ-VVL strains at 10^{th} and 30^{th} passages according to Example 2.

FIG. 11 shows neutralizing antibody titers and number of IFN-gamma secreting cells in piglets stimulated by BJ1805-2 and rBJ-VVL strains at 10^{th} and 30^{th} passages during immunization and challenge according to Example 2.

FIG. 12 shows viral loads in lung tissues of piglets after immunization and challenge with BJ1805-2 and rBJ-VVL strains at 10^{th} and 30^{th} passages according to Example 2.

FIG. 13 shows lung pathological anatomy, pathological sections, and immunohistochemistry results of piglets after challenge with BJ1805-2 and rBJ-VVL strains at 10^{th} and 30^{th} passages according to Example 2.

### DESCRIPTION OF EMBODIMENTS

The conventional experimental methods used in the following examples refer to the Molecular Cloning: A Laboratory Manual, Third Edition, By Sambrook et al. (Beijing: Science Press, 2002), and the use of instruments refers to their respective instrument operating instructions.

In the examples of the present disclosure, the viruses used include SD17-38 (GenBank Accession Number: MK689101), J1805-2 original strain (Chinese Patent Publication No. CN115992100A), rBJ1805-2 infectious clone strain (obtained by pACYC177-rBJ1805-2 plasmid rescue, Chinese Patent Publication No. CN115992100A), rBJ-VVL P10 passage strain (cultured in the present disclosure), rBJ-VVL P30 passage strain (cultured in the present disclosure), and rBJ-VVL P50 passage strain (cultured in the present disclosure). The cells used include BHK-21 cell line, Marc-145 cell line, and primary alveolar macrophages (PAMs, preserved in our laboratory)

In the examples of the present disclosure, plasmids and strains include plasmid pACYC177-rBJ1805-2, which is preserved in our laboratory (Chinese Patent Publication No. CN115992100A), and TOP10 competent cells, which are purchased from Beijing TransGen Biotech Co., Ltd.

In the examples of the present disclosure, the web-based tool used includes https://benchling.com.

In the examples of the present disclosure, other reagents used include: RNase-Free H₂O and trypsin cell digestion solution (phenol red), purchased from Solarbio; TRIpure Reagent for total RNA extraction, purchased from Aidlab Biotechnologies Co., Ltd; PrimeScript 1^{st} Strand cDNA Synthesis Kit, 2×PrimeSTAR MAX DNA Polymerase, and TAKARA Taq DNA Polymerase, purchased from Takara Bio Inc.; FastPure Plasmid Mini Kit purchased from Vazyme Biotech Co., Ltd.; One Step Clone Kit (homologous recombination reagent) purchased from Vazyme Biotech Co., Ltd.; DMEM culture medium purchased from HyClone Biochemical Products Co., Ltd.; fetal bovine serum purchased from Sigma Corporation; DyLight 594 Goat anti-Mouse IgG (H+L) secondary antibody purchased from Invitrogen; DNA Marker purchased from Zhejiang Biogene Science Co., Ltd.; 2×BioGold Tag Plus PCR MasterMix purchased from Zhejiang Biogene Science Co., Ltd.; Gel Extraction Kit purchased from Beijing ComWin Biotech Co., Ltd.; DNA restriction endonucleases purchased from Thermo Fisher Scientific; and T4 DNA Ligase and Lipofectamine™ 3000 Transfection Reagent purchased from Invitrogen.

### Example 1: Generation of In Vitro Cell-Adaptive Strain rBJ-VVL of Marc-145 and its Passage Strains Based on rBJ1805-2 Reverse Genetic Platform

1. Screening of Infectious Clone Virus rBJ1805-2 Adaptive to Marc-145 Cells Key Regions
1.1 Design of Primer Construction for Screening the Modified Straining with Key Amino Acid Sites Adaptive to Marc-145 Cells

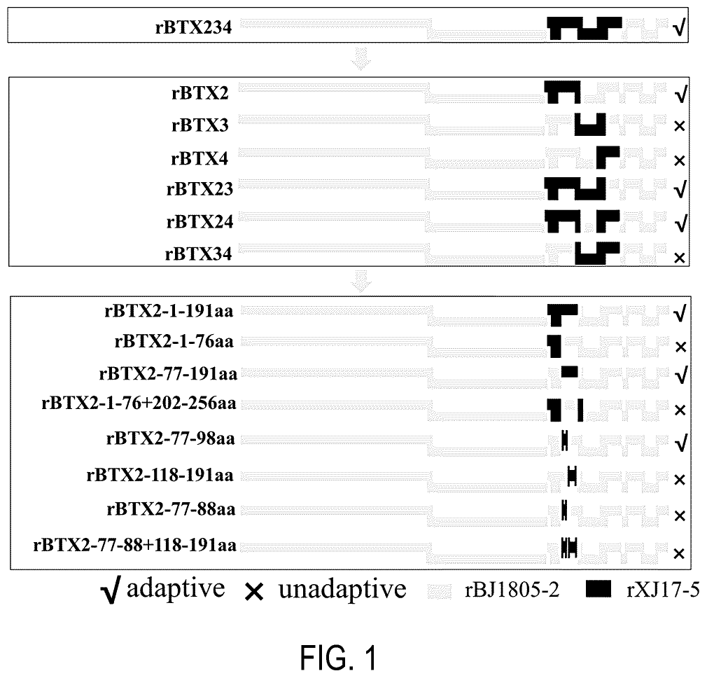

Nucleic acid sequences of pACYC177-rBJ1805-2 and the Marc-145-adaptive strain XJ17-5 (GenBank Accession Number: MK759853) were imported into an online platform https://benchling.com. Primers for constructing the modified strain in FIG. 1 were designed by sequence alignment. Detailed information on the construction of primers is shown in Table 1.

TABLE 1
Primers for screening key amino acid sites of infectious clone virus rBJ1805-2
adaptive to Marc-145 cells

| Name | Sequence information (5′-3′) | SEQ ID NO: |
|---|---|---|
| rBJ-XbaI-F3 | CGCTGCAATACTCATGGATAGTTGTGCTTGT | SEQ ID NO: 5 |
| rBJ-NOT1-2fu-1 | CAAACAACAGATGGCTGGCAACTAGAAGGCACAG | SEQ ID NO: 6 |
| rBTX234-ORF2-F | CAACGTTGGGCCTGGACTGAaatgaaatggggtctatgcaaagcc | SEQ ID NO: 7 |
| rBTX234-ORF1b- | ggctttgcatagaccccatttcattTCAGTCCAGGCCCAACGTTG | SEQ ID NO: 8 |
| R | | |
| rBTX234-ORF5-F | gccatcctactggcaatttgaATGTTCAGGTATGTTGG | SEQ ID NO: 9 |
| rBTX234-ORF4- | CCAACATACCTGAACATtcaaattgccagtaggatggc | SEQ ID NO: 10 |
| R | | |
| rBTX23-ORF3-F | ctcagtgccgcacggcgatagGAACACCCGTTTACATCA | SEQ ID NO: 11 |
| rBTX23-ORF3-R | TGATGTAAACGGGTGTTCctatcgccgtgcggcactgag | SEQ ID NO: 12 |
| rBTX4-ORF4-F | ggcaattggtttcacctggaATGGCTGCGTCCTTTC | SEQ ID NO: 13 |
| rBTX4-ORF4-R | GAAAGGACGCAGCCATtccaggtgaaaccaattgcc | SEQ ID NO: 14 |
| rBTX2-ORF3-F | gaactcatggtgaATTACACGGTGTGCCAG | SEQ ID NO: 15 |
| rBTX2-ORF3-R | CTGGCACACCGTGTAATtcaccatgagttc | SEQ ID NO: 16 |
| rBTX3-ORF3-F | CAGCAatggctaatagctgtacattcctcc | SEQ ID NO: 17 |
| rBTX3-ORF3-R | ggaggaatgtacagctattagccatTGCTG | SEQ ID NO: 18 |
| rBTX4-ORF4-F | GCAATTGGTTTCACCTAGAatggctacg | SEQ ID NO: 19 |
| rBTX4-ORF4-R | cgtagccatTCTAGGTGAAACCAATTGC | SEQ ID NO: 20 |
| rBTX2-1-191 aa-F | cagcaATGGCTAATAGCTGTACACTCCTC | SEQ ID NO: 21 |
| rBTX2-1-191 aa-R | GAGGAGTGTACAGCTATTAGCCATtgctg | SEQ ID NO: 22 |
| rBTX2-1-77 aa-F | caccctgagcaattacagaagatcttatgaGGTCTTC | SEQ ID NO: 23 |
| rBTX2-1-77 aa-R | GAAGACCtcataagatcttctgtaattgctcagggtg | SEQ ID NO: 24 |
| rBTX2-77-191 aa- | CTGAGCAGTTACAGAAGATCCTATGAggcctttc | SEQ ID NO: 25 |
| F | | |
| rBTX2-77-191 aa- | gaaaggccTCATAGGATCTTCTGTAACTGCTCAG | SEQ ID NO: 26 |
| R | | |
| rBTX2-77-98- | GTCAACCCTGATTGATGAAATGGTGTCGC | SEQ ID NO: 27 |
| 118-191 aa-F | | |
| rBTX2-77-98- | GCGACACCATTTCATCAATCAGGGTTGAC | SEQ ID NO: 28 |
| 118-191 aa-R | | |
| rBTX2-77-88 aa-F | gtgtcaggtagaTatccccaccTGGGGAACCAAGCAC | SEQ ID NO: 29 |
| rBTX2-77-88 aa-R | GTGCTTGGTTCCCCAggtggggatAtctacctgacac | SEQ ID NO: 30 |

### 1.2 Primer Pair Combination Method for Screening of Modified Viruses

Table 2 itemizes the combination methods of all primers shown in Table 1, as well as the plasmids obtained by PCR amplification and homologous recombination using the primer combinations. In Table 2, the product obtained by PCR amplification of a combination of primer 5 pair 1 is referred to as Modified Fragment 1, and the product obtained by PCR amplification of a combination of primer pair 2 is referred to as Modified Fragment 2.

TABLE 2
Primer pair combination for screening of modified viruses and resulting plasmids

| Modified | Name of | | Template | | Template | Resulting |
|---|---|---|---|---|---|---|
| strain | modified virus | | for primer | | for primer | modified |
| number | strain | Primer pair 1 | pair 1 | Primer pair 2 | pair 2 | plasmid |
| 1 | rBTX2 | rBJ-XbaI-F3 | pACYC177- | rBTX2-ORF3-F | pACYC177- | pACYC177- |
| | | and rBTX2- | rBTX234 | and rBJ-NOT1- | rBJ1805-2 | rBTX2 |
| | | ORF3-R | | 2fu-1 | | |
| 2 | rBTX23 | rBJ-XbaI-F3 | pACYC177- | rBTX23-ORF3-F | pACYC177- | pACYC177- |
| | | and rBTX23- | rBTX234 | and rBJ-NOT1- | rBJ1805-2 | rBTX23 |
| | | ORF3-R | | 2fu-1 | | |
| 3 | rBTX34 | rBJ-XbaI-F | pACYC177- | rBTX3-ORF3-F | pACYC177- | pACYC177- |
| | | and rBTX3- | rBJ1805-2 | and rBJ-NOT1- | rBTX234 | rBTX34 |
| | | ORF3-R | | 2fu-1 | | |
| 4 | rBTX4 | rBJ-XbaI-F | pACYC177- | rBTX4-ORF4-F | pACYC177- | pACYC177- |
| | | and rBTX4- | rBJ1805-2 | and rBJ-NOT1- | rBTX234 | rBTX4 |
| | | ORF4-R | | 2fu-1 | | |
| 5 | rBTX3 | rBJ-XbaI-F | pACYC177- | rBTX3-ORF3-F | pACYC177- | pACYC177- |
| | | and rBTX3- | rBJ1805-2 | and rBJ-NOT1- | rBTX23 | rBTX3 |
| | | ORF3-R | | 2fu-1 | | |
| 6 | rBTX24 | rBJ-XbaI-F | pACYC177- | rBTX4-ORF4-F | pACYC177- | pACYC177- |
| | | and rBTX4- | rBTX2 | and rBJ-NOT1- | rBTX4 | rBTX24 |
| | | ORF4-R | | 2fu-1 | | |
| 7 | rBTX2-1- | rBJ-XbaI-F | pACYC177- | rBTX2-1-191aa-F | pACYC177- | pACYC177- |
| | 191aa | and rBTX2-1- | rBTX2 | and rBJ-NOT1- | rBJ1805-2 | rBTX2-1- |
| | | 191aa-R | | 2fu-1 | | 191aa |
| 8 | rBTX2-77- | rBJ-XbaI-F | pACYC177- | rBTX2-77-191aa- | pACYC177- | pACYC177- |
| | 191aa | and rBTX2- | rBTX2-1- | F and rBJ-NOT1- | rBJ1805-2 | rBTX2-77- |
| | | 77-191aa-R | 191aa | 2fu-1 | | 191aa |
| 9 | rBTX2-1-76aa | rBJ-XbaI-F | pACYC177- | rBTX2-1-77aa-F | pACYC177- | pACYC177- |
| | | and rBTX2-1- | rBTX2-1- | and rBJ-NOT1- | rBJ1805-2 | rBTX2-1-76aa |
| | | 77aa-R | 191aa | 2fu-1 | | |
| 10 | rBTX2-1-76 + | rBJ-XbaI-F | pACYC177- | rBTX2-77-98- | pACYC177- | pACYC177- |
| | 202-256aa | and rBTX2- | rBTX2-1- | 118-191aa-F and | rBTX2 | rBTX2-1-76 + |
| | | 77-98-118- | 76aa | rBJ-NOT1-2fu-1 | | 202-256aa |
| | | 191aa-R | | | | |
| 11 | rBTX2-77- | rBJ-XbaI-F | pACYC177- | rBTX2-77-98- | pACYC177- | pACYC177- |
| | 98aa | and rBTX2- | rBTX2-77- | 118-191aa-F and | rBJ1805-2 | rBTX2-77- |
| | | 77-98-118- | 191aa | rBJ-NOT1-2fu-1 | | 98aa |
| | | 191aa-R | | | | |
| 12 | rBTX2-118- | rBJ-XbaI-F | pACYC177- | rBTX2-77-98- | pACYC177- | pACYC177- |
| | 191aa | and rBTX2- | rBJ1805-2 | 118-191aa-F and | rBTX2- | rBTX2-118- |
| | | 77-98-118- | | rBJ-NOT1-2fu-1 | 77-191aa | 191aa |
| | | 191aa-R | | | | |
| 13 | rBTX2-77- | rBJ-XbaI-F | pACYC177- | rBTX2-77-88aa-F | pACYC177- | pACYC177- |
| | 88aa | and rBTX2- | rBTX2-77- | and rBJ-NOT1- | rBJ1805-2 | rBTX2-77- |
| | | 77-88aa-R | 191aa | 2fu-1 | | 88aa |
| 14 | rBTX2-77-88 + | rBJ-XbaI-F | pACYC177- | rBTX2-77-98- | pACYC177- | rBTX2-77-88 + |
| | 118-191aa | and rBTX2- | rBTX2-77- | 118-191aa-F and | rBTX2- | 118-191aa |
| | | 77-98-118- | 88aa | rBJ-NOT1-2fu-1 | 118-191aa | |
| | | 191aa-R | | | | |

### 1.3 Construction Method of Modified Cloning Plasmid

pACYC177-rBTX234 was first constructed, and its schematic structure replacement diagram was shown in FIG. 1. The construction method was as follows: rBJ-XbaI-F3 and rBTX234-ORF1b-R were combined, rBTX234-ORF2-F and rBTX234-ORF4-R were combined, rBTX234-ORF5-F and rBJ-NOT1-2fu-1 were combined, and rBTX234-1 segment, rBTX234-2 segment and rBTX234-3 segment were obtained by PCR amplification. The pACYC177-rBTX234 modified cloning plasmid was then obtained by homologous recombination.

The PCR amplification method and the homologous recombination method used for obtaining the modified fragments are as follows:

### 1.3.1 PCR Amplification Method and System for Modified Fragments

In the present disclosure, all modified fragments were amplified using the following PCR amplification method. The system and procedure of PCR amplification are shown in Table 3 and Table 4. The commercial kit was 2×PrimeSTAR MAX DNA Polymerase (purchased from Takara Bio Inc.).

TABLE 3
Reaction system

| | | | 2 × PrimeSTAR | |
|---|---|---|---|---|
| | | Forward and | MAX DNA | RNase |
| Name | Template | reverse primers | Polymearse | Free H₂O |
| Dose | 2 μL | 1 μL each | 20 μL | Make up |
| | | | | to 40 μL |

TABLE 4
Reaction procedure

| | | | | | | | Number of |
|---|---|---|---|---|---|---|---|
| Step | Denaturation | | Annealing | | Extension | | cycles |
| Temperature | 98° | C. | 50° | C. | 72° | C. | 35 cycles |
| Time | 10 | s | 30 | s | 2 | min | |

The primer pair combinations in Table 2 were combined using the above method, the Modified Fragment 1 and the Modified Fragment 2 required for each modified strain shown in FIG. 1 could be obtained. Electrophoresis was performed for 30 min using a 1% agarose gel with large holes, enzyme digestion bands were separated by agarose gel electrophoresis, target bands were excised for gel recovery, and the amplified modified fragments were stored at −20° C. 1.3.2 Vector digestion and homologous recombination method and system

All vector digestion and homologous recombination methods in the present disclosure were performed as follows. First, the reverse genetics platform plasmid rBJ1805-2 was double-digested with Xba I and Not I to prepare a linearized vector pACYC177-BJ1805-2-F1+F2. A specific reaction system for the double digestion is shown in Table 5.

TABLE 5
Double-digestion system for pACYC177-rBJ1805-2 plasmid

| | pACYC177- | | | 10 × | |
|---|---|---|---|---|---|
| | rBJ1805-2 | | | CutSmart | RNase |
| Name | plasmid | Xba I | Not I | Buffer | Free H₂O |
| Dose | 3000 ng | 3 μL | 3 μL | 4 μL | Make up |
| | | | | | to 40 μL |

The digestion was carried out at 37° C. for 30 min according to the above system. Enzyme digestion bands were separated by agarose gel electrophoresis, target bands were excised for gel recovery. The pACYC177-BJ1805-2-F1+F2 linearized vector had a concentration of 30 ng/μL upon measurement, and stored at −20° C.

The Modified Fragment 1 and the Modified Fragment 2 recovered from the gel homologously recombined with the linearized vector pACYC177-BJ1805-2-F1+F2 (One Step Clone Kit). A specific reaction system is shown in Table 6.

TABLE 6
Homologous recombination system

| | | | pACYC177- | 5 × CE | | |
|---|---|---|---|---|---|---|
| | Modified | Modified | BJ1805-2- | MultiS | Exanse | |
| Name | Fragment 1 | Fragment 2 | F1 + F2 | Buffer | MultiS | H₂O |
| Dose | 50 ng | 50 ng | 200 ng | 2 μL | 1 μL | To 10 μL |

The reaction system was reacted at 37° C. for 30 min, then immediately placed on ice, and transformed into TOP10 super competent cells; independent colonies were picked for pure culture, and bacterial liquid PCR detection was performed using detection primers. PCR positive bacteria were selected and cultured overnight for plasmid extraction, the extracted plasmids were digested with Xba I and Asc I, and the digested products were subjected to electrophoresis on a 0.9% agarose gel, a correct length of bands under enzyme digestion was 6061 bp, and the plasmid with a correct size was selected and kept.

### 1.4 Rescue of Modified Virus in PAM Cells and its Cell Adaptability Test of Marc-145

### 1.4.1 PAM Cell Rescue of Modified Cloning Plasmid

BHK-21 cells were pre-seeded into a 24-well cell culture plate at a density of 2.5×10⁵ cells/well using DMEM culture medium containing 10% FBS, and cultured in a 37° C., 5% CO₂ incubator until a cell density reached about 80%. Cell transfection was performed according to the instructions of Lipofectamine™ 3000 Transfection Reagent.

Transfection method: first, 2 μg of plasmid, 4 μL of P3000 plasmid, and 50 μL of OPTI-MEM reagent were mixed to prepare Solution A; 3 μL of Lip 3000 and 50 μL of DMEM were mixed to prepare Solution B; Solution A and Solution B were then combined and incubated at 25° C. for 15 min to obtain a mixture; and the mixture was then added to the BHK-21 cells. The transfection system is shown in Table 7.

48 h after the cell transfection, the 12-well plate was sealed and frozen at −80° C., and subjected to repeated freezing and thawing operations twice, all cell suspension was taken and centrifuged at 10,000×g for 4 min, and a transfection supernatant was collected.

TABLE 7
Transfection system

| Type/name | Plasmid | Lip3000 | P3000 | DMEM |
|---|---|---|---|---|
| Plasmid | 2000 ng | — | 3 μL | 50 μL |
| premixed solution | | | | |
| Lip3000 | — | 4 μL | — | 50 μL |
| premixed solution | | | | |

Primary PAM cells were pre-inoculated with 2 mL of RPMI (1640) culture medium containing 2% FBS at 2×10⁵ cells/well in a 12-well cell culture plate and cultured at 37° C. and 5% CO₂. After the primary PAM cells were adhered to a wall, 500 μL of the transfection supernatant was taken and covered on the primary PAM cells, and incubated for 2 h, and a supernatant was discarded, and RPMI (1640) culture medium containing 2% FBS was added for continuous culture. In addition, primary PAM cells were infected with the parental virus BJ1805-2 as a positive control, and cultured for 3-4 days, and cytopathic effects of PAM cells were observed. A cell supernatant was collected, cells were left, and identified for IFA using PRRSV-N protein-specific monoclonal antibody 6A1.

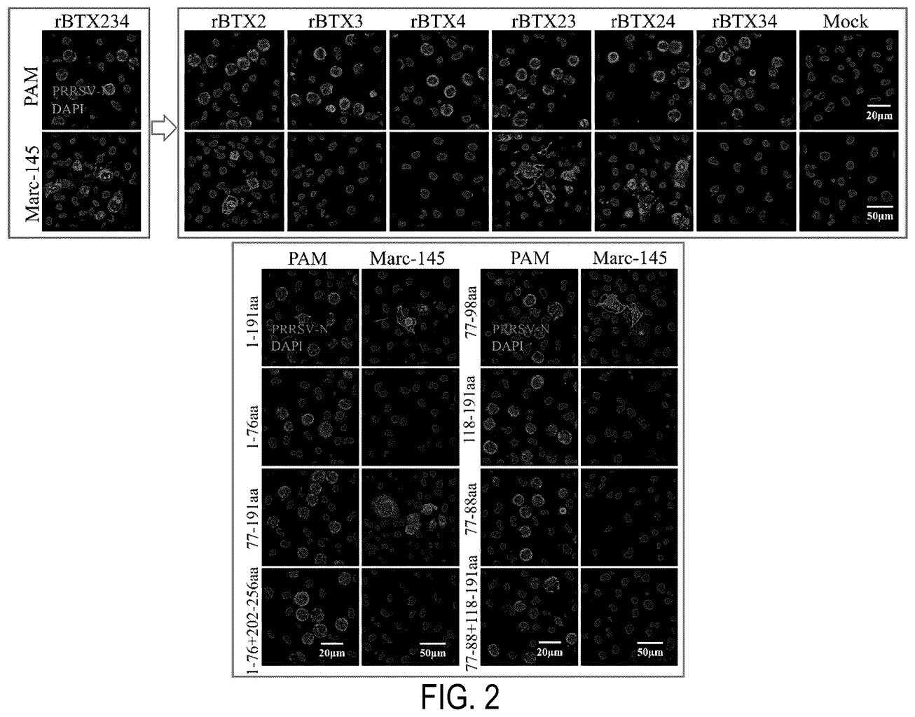

Rescue results of PAM cells were shown in FIG. 2. After IFA identification, all modified viruses constructed in FIG. 1 exhibited specific red fluorescence, indicating that all modified viruses constructed in FIG. 1 could be successfully rescued in PAM cells.

### 1.4.2 Adaptability Test and Serial Passage of Successfully Rescued Virus Solution in Marc-145 Cells

Marc-145 cells were pre-inoculated into a 12-well cell culture plate at a density of 2×10⁵ cells/well using DMEM culture medium containing 10% FBS, and cultured in a 37° C., 5% CO₂ incubator until a cell density reached about 80%, and the culture medium was then replaced with DMEM containing 2% FBS.

500 μL of the virus solution successfully rescued in PAM cells described in Section 1.4.1 was taken and inoculated into Marc-145 cells. The virus strain was serially passaged blindly for 5 generations, the 5^{th} generation modified virus was taken and inoculated into Marc-145 cells, and IFA identification was performed at 144 hours post infection (hpi) using the PRRSV-2 N-specific monoclonal antibody 6A1.

As shown in FIG. 2, IFA identification results show that rBTX2, rBTX23, rBTX24 and rBTX234 can detect specific red fluorescence in Marc-145 cells, indicating that rBTX2, rBTX234, rBTX23 and rBTX24 can infect Marc-145, while rBTX3, rBTX4 and rBTX34 cannot infect Marc-145 cells. The construction strategy is shown in FIG. 1. The strains adaptive to Marc-145 cells all replaced ORF2, indicating that the key amino acid site reside in GP2a protein encoded by the ORF2 gene.

GP2a had a full length of 256 amino acids (aa), with a specific structure as follows: a region overlapping with GP3 is 202-256aa; an identical region between rBJ1805-2 and XJ17-5 is 191-202aa; and a variable region between rBJ1805-2 and XJ17-5 is 1-191aa. IFA identification results are shown in FIG. 2. Among the three modified viruses, that is, rBTX2-1-191aa, rBTX2-1-76aa and rBTX2-77-191aa constructed according to the identical region and the variable region, rBTX2-1-191aa and rBTX2-77-191aa could be adaptive to Marc-145 cells, while rBTX2-1-76aa could not be adaptive to Marc-145 cells. Subsequently, 1-76+202-256aa was combined and replaced to verify whether the GP3 overlapping region was involved in the culture of Marc-145 cells. IFA identification results showed that rBTX2-1-76+202-256aa could not infect Marc-145 cells. In summary, 77-191aa of GP2a protein encoded by ORF2 gene was a minimum range for rBJ1805-2 to adapt to Marc-145 cells.

For the 77-191aa of GP2a protein encoded by ORF2 gene, variable regions were 77-98aa and 118-191aa, and 99-117 was identical sequence. rBTX2-77-98aa and rBTX2-118-191aa were constructed based on the differential and similar sequences. As shown in FIG. 2, IFA identification results indicated that only rBTX2-77-98aa could infect Marc-145 cells. The further constructed rBTX2-77-88aa and rBTX2-77-88aa+118-191aa were unable to infect Marc-145 cells. In summary, the remaining amino acids 89-98aa were the key region determining the adaptation of rBJ1805-2 to Marc-145 cells.

1.5 Serial Passage of Modified Viruses Infected with Marc-145 Cells in Marc-145 Cells

Marc-145 cells were pre-inoculated into a 12-well cell culture plate at a density of 2×10⁵ cells/well using DMEM culture medium containing 10% FBS, and cultured in a 37° C., 5% CO₂ incubator until a cell density reached about 80%, and the culture medium was then replaced with DMEM containing 2% FBS.

The modified virus liquid that successfully adapted to Marc-145 cells described in Section 1.4.2 were inoculated into fresh Marc-145 cells at an MOI of 0.01. 2 h after the virus adsorbed, the cells were washed three times with PBS, and the culture medium was then replaced with DMEM containing 2% FBS until 144 hpi. After the cytopathic effects were observed, freezing and thawing operations were repeated, and the virus was repeatedly passaged to a tenth generation and stored in a −80° C. refrigerator for subsequent use.

### 1.6 Determination of Growth Characteristics of Virus Strains Adaptive to Marc-145 Cells

First, a TCID₅₀ titer of each adaptive strain of the tenth generation was determined. A determination method of the TCID₅₀ titer was as follows: Marc-145 cells were pre-inoculated into a 96-well cell culture plate at a density of 4×10⁴ cells/well using DMEM culture medium containing 10% FBS, and cultured in a 37° C., 5% CO₂ incubator until a cell density reached about 80%, and the culture medium was then replaced with DMEM containing 2% FBS. A virus stock solution was taken and serially diluted by 10-fold, 100-fold, 1,000-fold, 10,000-fold, 100,000-fold, and 1,000,000-fold, respectively. The solution at each dilution gradient was inoculated into 96 wells with 4 replicate wells, and 2 h after the virus adsorbed, the culture medium was replaced with 200 μL fresh DMEM containing 2% FBS. At 144 hpi, the wells with cytopathic effects were counted, and TCID₅₀ values of the virus stock solutions were calculated, respectively. Specific titers of each adaptive virus stock solution are shown in Table 8.

TABLE 8
Titers of virus stock solutions of modified viruses

| Name of modified | Virus titer |
|---|---|
| virus strain | (TCID₅₀/mL) |
| rBTX234 | 10^{4.3} |
| rBTX2 | 10^{3.769} |
| rBTX23 | 10^{4.3} |
| rBTX24 | 10⁴ |
| rBTX2-1-191aa | 10³ |
| rBTX2-77-191aa | 10³ |
| rBTX2-77-98aa | 10^{2.5} |

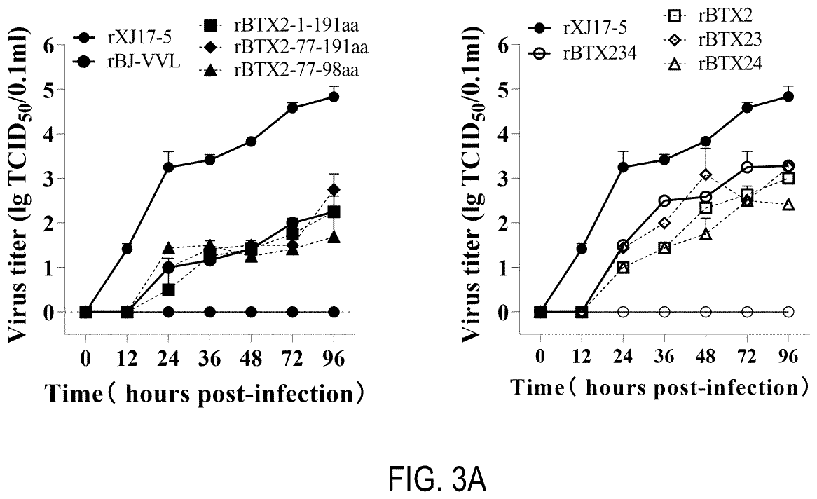

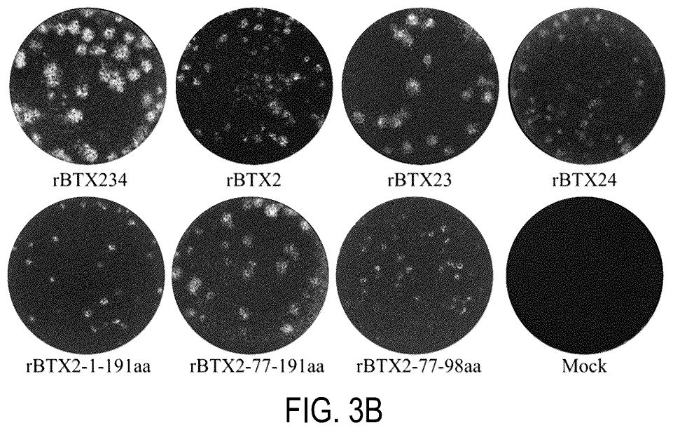

The replication characteristics of various adaptive viruses in Marc-145 cells were evaluated by plotting multi-step growth curves. The specific method was as follows: each virus solution was inoculated onto Marc-145 cells cultured in 6-well plate at an MOI of 0.01. Supernatants at six time points, that is, 12 hpi, 24 hpi, 36 hpi, 48 hpi, 72 hpi, and 96 hpi, were collected and placed in a −80° C. refrigerator for frozen storage. After the supernatants at all the time points were collected, titers of the supernatants at the different time points were determined according to the TCID₅₀ titer determination method, and standard curves were then plotted. Results of the multi-step growth curves were shown in FIG. 3A. All modified virus strains were capable of completing proliferation on Marc-145 cells.

### 1.7 Plaque Assay of Adaptive Viruses in Marc-145 Cells

Marc-145 cells were pre-inoculated into a 24-well cell culture plate at a density of 1×10⁵ cells/well using DMEM culture medium containing 10% FBS, and cultured in a 37° C., 5% CO₂ incubator until a cell density reached about 80%, and the culture medium was then replaced with DMEM containing 2% FBS. A cell density was observed the next day, and the next step was performed until reached a dense state.

A virus stock solution was serially diluted by 10-fold, 100-fold, 1,000-fold, 10,000-fold, 100,000-fold, and 1,000,000-fold, respectively, and then infected Marc-145 cells for 2 h. The virus solution was discarded after 2 h, the cells were gently washed three times with 1×PBS, a 0.7% agarose culture medium with a low melting point was prepared (a formulation of 30 mL culture medium: 15 mL DMEM, 600 μL FBS, and 300 μL penicillin-streptomycin solution were mixed to obtain a mixture, and 15 mL of 1.4% agar solution with a low melting point was finally added into the mixture), and after cooling about 37° C., a culture medium was directly applied to monolayer cells. The 6-well plates were then inverted for culture, and observed after 6 days. After cytopathic effects were observed, the culture medium was covered with 4% paraformaldehyde, and fixed overnight and then stained with crystal violet for 1 h. After staining, the cells were rinsed with water and observed. Results of the plaque assay were shown in FIG. 3B. All the adaptive viruses were able to produce distinct plaques on Marc-145 cells.

2. Rescue of rBJ-VVL Virus and Verification of Reverse Mutation at Three Amino Acid Points of GP2a-91/97/98
2.1 Construction, Rescue and Marc-145 Adaptability Test of rBJ-VVL Virus

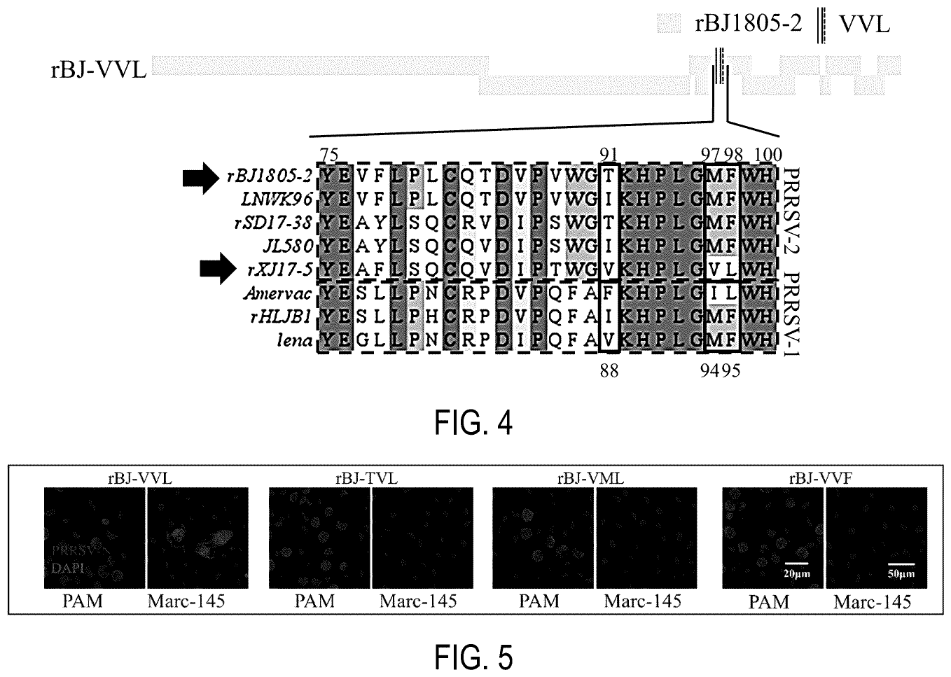

Based on the results of final screening described in Section 1, the 89-98aa sequences of rBJ1805-2 and XJ17-5 GP2a were compared. The sequence comparison results were shown in FIG. 4. The comparison results shown that differences were observed at positions 91, 97, and 98 in the 88-98aa of PRRSV strain GP2a. Therefore, the amino acids at positions 91, 97, and 98 of GP2a were the key amino acid sites for rBJ1805-2 to adapt to Marc-145 cells. Three point mutations were performed on the three sites.

Using the Benchling online tool, a comparison analysis was performed based on the plasmid sequence of the reverse genetics platform pACYC177-rBJ1805-2 (sequence composition shown in SEQ ID NO: 1+SEQ ID NO: 2) and the Marc-145 cell-adaptive strain XJ17-5, as shown in FIG. 1. The amino acid coding sequence of “TMF” at positions GP2a-91/97/98 of rBJ1805-2 was replaced with the “VVL” pattern at positions GP2a-91/97/98 of XJ17-5. The designed primers were used for homologous recombination, requiring a GC content of 40%-60% and a minimum length of ≥18 bp. Specifically, the specific modified primers included the aforementioned two modified primers and two modified primers fixed on the vectors of the reverse genetics platform. Details of the primers are shown in Table 9.

TABLE 9
rBJ-VVL mutation primers

| Primer | | SEQ ID |
|---|---|---|
| name | Sequence (5′-3′) | NO: |
| rBJ-VVL-F | CGTCTGGGGAgtCAAGCACCCCCTGGGA | SEQ ID |
| | gTGcTTTGGCACCACAAG | NO: 3 |
| rBJ-VVL-R | CTTGTGGTGCCAAAgCAcTCCCAGGGGG | SEQ ID |
| | TGCTTGacTCCCCAGACG | NO: 4 |

The primer pair rBJ-VVL-R and rBJ-XbaI-F were combined, the primer pair rBJ-VVL-F and rBJ-NOT1-2fu-1 were combined, and the Modified Fragment 1 and Modified Fragment 2 were obtained according to the PCR amplification method described in Section 1.3.1. The modified clone plasmid pACYC177-rBJ-VVL was obtained according to the homologous recombination method described in Section 1.3.2. The pACYC177-rBJ-VVL modified cloning plasmid was constructed, the modified virus PAM cells were rescued and Marc-145 adaptive culture test were performed according to the method described in Section 1.4. As shown in FIG. 5, IFA results indicated that rBJ-VVL could successfully infect Marc-145 cells.

The rBJ-VVL virus strain was serially passaged 10, 30, and 50 generations according to the, method described in Section 1.5 to obtain rBJ-VVL P10, rBJ-VVL P30, and rBJ-VVL P50, respectively.

The TCID₅₀ titers of the viral passages were determined according to the method described in Section 1.6. The titers were as follows: rBJ-VVL P10: 10^{2.5} TCID₅₀/mL, rBJ-VVL P30: 10^{6.3} TCID₅₀/mL; and rBJ-VVL P50: 10^{6.9} TCID₅₀/mL.

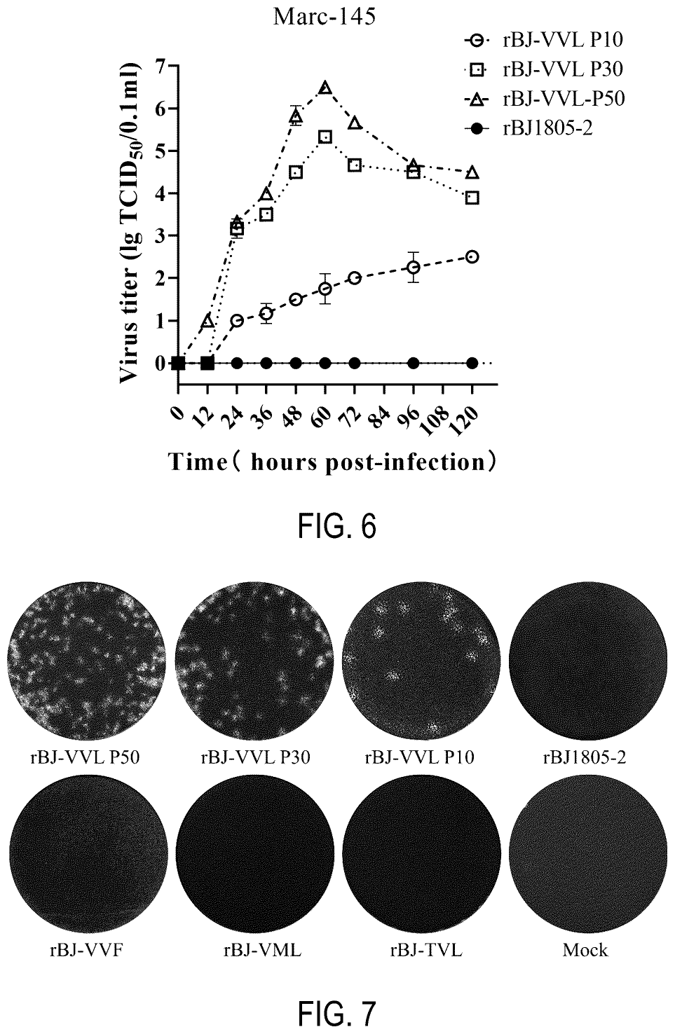

Multi-step growth curves of rBJ-VVL P10, rBJ-VVL P30 and rBJ-VVL P50 strains were generated according to the method described in Section 1.7, and results were shown in FIG. 6. The growth ability of rBJ-VVL P30 and rBJ-VVL P50 on Marc-145 was significantly higher than that of rBJ-VVL P10.

Plaque assays were conducted on rBJ-VVL P10, rBJ-VVL P30 and rBJ-VVL P50 strains according to the method described in Section 1.8, and results were shown in FIG. 7. A number of plaques in the 1000^{th} dilution of rBJ-VVL P30 and rBJ-VVL P50 was significantly higher than that of rBJ-VVL P10. The above results showed that rBJ-VVL P30 and rBJ-VVL P50 could produce a large amount of fresh progeny virus fluid.

2.2 Verification of rBJ-TVL, rBJ-VML and rBJ-VVF Reverse Mutant Strains

In order to confirm the necessity of the amino acid sites at positions 91/97/98 for the adaptation of rBJ1805-2 to Marc-145 cells, and reverse mutation verification was performed on each site. Specific primers are shown in Table 10.

TABLE 10
Primers for reverse mutation

| | | SEQ ID |
|---|---|---|
| Name | Sequence (5′-3′) | NO: |
| rBJ-VVL- | CGTCTGGGGAACCAAGCACCCCCTGGGAgTGc | SEQ ID |
| V91T-F | TTTGGCACCACAAG | NO: 31 |
| rBJ-VVL- | CTTGTGGTGCCAAAgCAcTCCCAGGGGGTGCT | SEQ ID |
| V91T-R | TGGTTCCCCAGACG | NO: 32 |
| rBJ-VVL- | CGTCTGGGGAgtCAAGCACCCCCTGGGAATGc | SEQ ID |
| V97M-F | TTTGGCACCACAAG | NO: 33 |
| rBJ-VVL- | CTTGTGGTGCCAAAgCATTCCCAGGGGGTGCT | SEQ ID |
| V97M-R | TGacTCCCCAGACG | NO: 34 |
| rBJ-VVL- | CGTCTGGGGAgtCAAGCACCCCCTGGGAgTGT | SEQ ID |
| L97F-F | TTTGGCACCACAAG | NO: 35 |
| rBJ-VVL- | CTTGTGGTGCCAAAACACTCCCAGGGGGTGCT | SEQ ID |
| L97R-R | TGacTCCCCAGACG | NO: 36 |

The primers rBJ-XbaI-F3, rBJ-VVL-V91T-F, rBJ-VVL-V97M-F and rBJ-VVL-L97F-F was combined with rBJ-NOT1-2fu-1, respectively, to amplify downstream fragments of rBJ-TVL, rBJ-VML and rBJ-VVF mutants. rBJ-XbaI-F3 was combined with rBJ-VVL-V91T-R, rBJ-VVL-V97M-R and rBJ-VVL-L97R-R, respectively, to amplify upstream fragments of rBJ-TVL, rBJ-VML and rBJ-VVF mutants. The mutant strains rBJ-TVL, rBJ-VML and rBJ-VVF were obtained according to the PCR amplification method and the homologous recombination method described in Section 1.3. The rescue of rBJ-TVL, rBJ-VML and rBJ-VVF in PAM cells and adaptability test of the same to Marc-145 cells were conducted according to the described in Section 1.4.

Results are shown in FIG. 5. Indirect immunofluorescence assay showed that rBJ-TVL, rBJ-VML, and rBJ-VVF were all successfully rescued in PAM cells. However, no specific fluorescence signal was detected in Marc-145 cells inoculated with rBJ-TVL, rBJ-VML, or rBJ-VVF. The results indicate that the V/V/L amino acid motif is essential for NADC34-like PRRSV-2 to obtain tropism for Marc-145 cells.

According to the above findings, it is proved that, apart from the modified viruses that are successfully adaptive to Marc-145 cell culture as described in Section 1, the V/V/L motif is a set of key amino acid sites that determine the adaptation of rBJ1805-2 to Marc-145 cell culture.

### Example 2 Piglet Immunization and Virus Challenge Test of rBJ1805-2, rBJ-VVL P10, and P30

1. Piglet Immunization and Virus Challenge of rBJ1805-2, rBJ-VVL P10, and rBJ-VVL P30

20 4-week-old piglets, negative for pseudorabies virus (PRV), porcine circovirus (PCV), porcine epidemic diarrhea virus (PEDV) and other important pathogens, were selected from a pig farm. 5 piglets were inoculated with 2 mL of rBJ1805-2 virus solution containing 10⁵ TCID₅₀ another 5 piglets were respectively inoculated with 2 mL of rBJ-VVL P10 and rBJ-VVL P30 virus solution, each containing 10⁵ TCID₅₀, 3 additional piglets were inoculated with 2 mL of DMEM culture medium, and The remaining 2 piglets were taken as negative controls. All immunizations were administered via intramuscular injection.

After 42 days of immunization and complete clearance of viremia in the piglets, the challenge test was carried out. Each piglet was intranasally challenged with 2 mL of NADC30-like SD17-38 wild-type strain (the strain was isolated and preserved in our laboratory, GenBank Accession Number MK689101) containing 10⁵ TCID₅₀.

### 2. Monitoring of Viremia, Body Temperature and Weight of Piglets

Blood samples were collected at the time points shown in FIG. 6, and viral loads in the samples were detected by qRT-PCR. Specifically, serum samples were collected in coagulation-promoting tubes and incubated at 37° C. for 30 min to promote blood coagulation. The samples were then centrifuged at 4000 rpm for 30 min. 200 μL of supernatant was taken and used to extract total RNA using the TRIpure Reagent (purchased from Aidlab Biotechnologies Co., Ltd), and the extracted total RNA was stored at −20° C. for short-term preservation or −80° C. for long-term preservation. The viral RNA was reverse-transcribed into cDNA using a reverse transcription kit (PrimeScript™ 1^{st} strand cDNA Synthesis Kit, TAKARA), and the resulting cDNA was stored at −20° C. for subsequent use. The reaction system and procedures are shown in Tables 11 and 12, respectively.

TABLE 11
Total RNA preprocessing system and procedures

| | OligodT | Random | dNTP | |
|---|---|---|---|---|
| | Primer | 6 mers | Mixture | Template |
| Name | (50 μM) | (50 μM) | (10 mM each) | RNA |
| Dose | 1 μL | 0.4 μL | 1 μL | 7.6 μL |

| | |
|---|---|
| Procedure | 65° C., 5 min |

TABLE 12
Total RNA reverse transcription system and procedures

| | Prepro- | 5 × | RNase | PrimeScript | RNase |
|---|---|---|---|---|---|
| | cessing | PrimeScript | Inhibitor | RTase | free |
| Name | products | Buffer | (40 U/μL) | (200 U/μL) | d H₂O |
| Dose | 10 μL | 0.4 μL | 1 μL | 7.6 μL | Make up |
| | | | | | to 20 μL |

| | |
|---|---|
| Procedures | at 30° C. for 10 min; at 42° C. for |
| | 60 min; and at 70° C. for 15 min |

TABLE 13
qPCR system and procedures

| | | | Primer (forward | | |
|---|---|---|---|---|---|
| | | | and reverse | | |
| | | | primers are both | | |
| | | | 10 μM mixed | Taq | |
| Name | cDNA | Probe | solution) | enzyme | H₂O |
| Dose | 1 μL | 0.4 μL | 0.5 μL | 10 μL | 8.1 μL |

| | |
|---|---|
| Procedures | at 98° C. for 10 s, at 60° C. |
| | for 10 s, and repeated for 40 cycles |

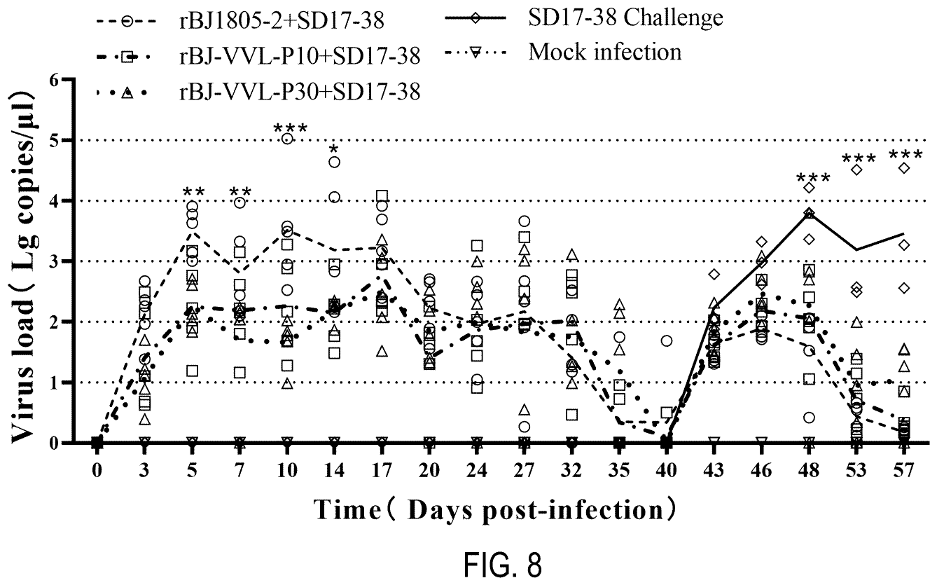

The qPCR system and procedures are shown in Table 13. 1 μL of cDNA, 0.4 μL of probe, 0.5 μL of primer mix (forward and reverse primers were pre-mixed at a working concentration of 10 μM each), 10 μL of Taq enzyme (purchased from TAKARA), and 8.1 μL of H₂O were taken and regarded as a monitoring well. Probe and primer information are shown in Table 14, wherein FAM and MGB represent fluorescent markers, not a part of the sequence. 3 replicate wells were set for each sample. The procedures were as follows: at 98° C. for 10 s, at 60° C. for 10 s, and repeated for 40 cycles, and signal acquisition was performed after each cycle. qPCR was used to monitor viremia in the vaccinated piglets. As shown in FIG. 8, the viremia of piglets immunized with the passaged rBJ-VVL P10 and P30 strains was significantly lower than that immunized with the parental strain rBJ1805-2, indicating that it could provide protective efficacy against the SD17-38 wild-type virus, and reduce viremia rapidly.

TABLE 14
[]qPCR probe and primer information

| | Sequence information | SEQ ID |
|---|---|---|
| Name | (5′-3′) | NO: |
| PRRSV2N34-P | FAM-TGTGAGCACCGTTTAT- | SEQ ID |
| | MGB | NO: 37 |
| NADC34-F13240 | TGGTTGGCGTTCTTGTCCTT | SEQ ID |
| | | NO: 38 |
| NADC34-R13340 | CATCATGAACGGCACAAATGA | SEQ ID |
| | | NO: 39 |

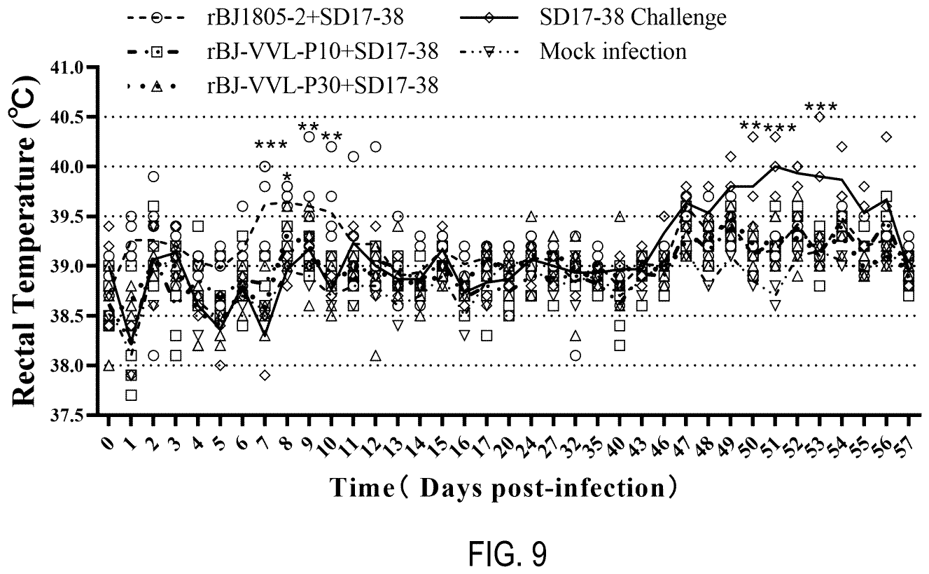

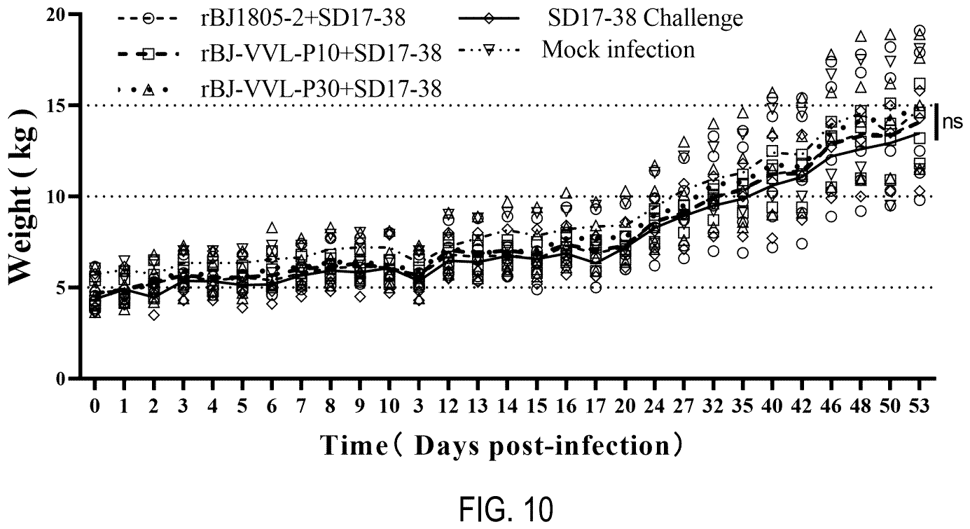

Body temperature and weight were measured at the time points indicated in FIGS. 9 and 10. To measure the temperature, a thermometer was inserted into the anuses and left in place until the reading stabilized. As shown in FIGS. 9 and 10, the temperatures of piglets immunized with the passaged rBJ-VVL P10 and P30 strains was significantly lower than those immunized with the parental strain rBJ1805-2. After immunization with rBJ1805-2, rBJ-VVL P10 and P30, they were able to provide protection against wild-type SD17-38 and keep the body temperature of immunized piglets stable. The body weight of piglets in all groups after the challenge is shown in FIG. 10, with no significant difference.

### 3. Analysis of Protective Immune Response in Piglets

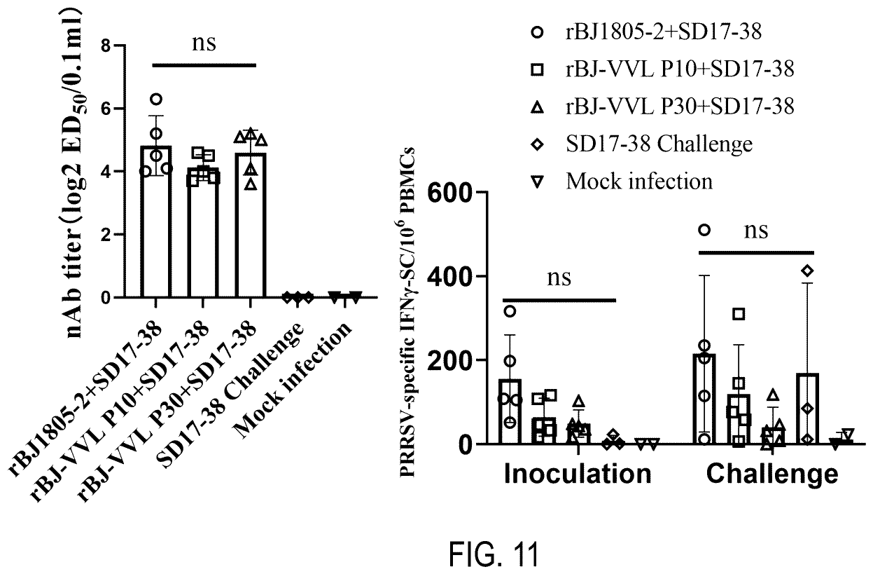

In order to determine the protective humoral immune response, serum samples were collected on Day 14 after challenge for neutralization test. The specific method was as follows: Marc-145 cells were pre-inoculated into a 24-well cell culture plate at a density of 1×10⁵ cells/well using DMEM culture medium containing 10% FBS, and cultured in a 37° C., 5% CO₂ incubator until a cell density reached about 80%, and the culture medium was then replaced with DMEM containing 2% FBS. The collected serum samples were first inactivated at 56° C. for 30 min, then serially diluted at ratios of 1:8, 1:16, 1:32, and 1:64, a total volume of the serum dilution system was 50 μL. A 50 μL of virus solution containing 50 TCID₅₀ was mixed with each diluted serum sample and placed in an incubator at 37° C. and 5% CO₂ for 1.5 h. The mixed solution was then transferred into a 96-well plate pre-seeded with Marc-145 cells. 4 replicate wells were set up for each serum dilution gradient. Titers of neutralizing antibodies were calculated using a TCID₅₀ algorithm to obtain a Log₂ value of the dilution factor. A specific dilution ratio could be obtained by calculating according to a POWER (2, Log₂ value) formula in an EXCEL table. As shown in FIG. 11A, the results indicated that the piglets in the rBJ1805-2, rBJ-VVL P10 and P30 immunization groups all produced high titers of neutralizing antibodies, and there was no statistically significant difference in the titers of neutralizing antibodies among the groups.

In addition, in order to determine the protective cellular immune response, levels of PRRSV-specific IFN-γ-secreting cells in PBMCs collected on Day 42 after immunization and Day 14 after challenge were determined. As shown in FIG. 11B, the results showed that after immunization with rBJ1805-2, rBJ-VVL P10 and P30, PRRSV-specific IFN-γ secreting cells were induced, and there was no significant difference among the immunized groups. After challenge, the levels of PRRSV-specific IFN-γ-secreting cells in the immunized and challenged groups increased; however, compared with the challenged-only group, the differences were not statistically significant. This may be attributed to the substantial genomic differences between the challenge strain and the immune strain (only 80.39% similarity across the full genome), resulting in the absence of a significant memory IFN-γ-secreting cell response.

### 4. Detection of Viral Load in Lung Tissues of Piglets

1 g of lung lobule sample was first collected from each piglet, 1 mL of PBS was added and ground for 3 min to obtain a homogenate, the homogenate was centrifuged at 5000 rpm for 5 min, and 200 μL of supernatant was taken to extract RNA. The extracted lung RNA of each piglet was quantified, and an RNA concentration was recorded. RNA reverse transcription was subsequently performed to obtain the lung tissue cDNA. Specific method and system for obtaining the cDNA are described in Section 2.

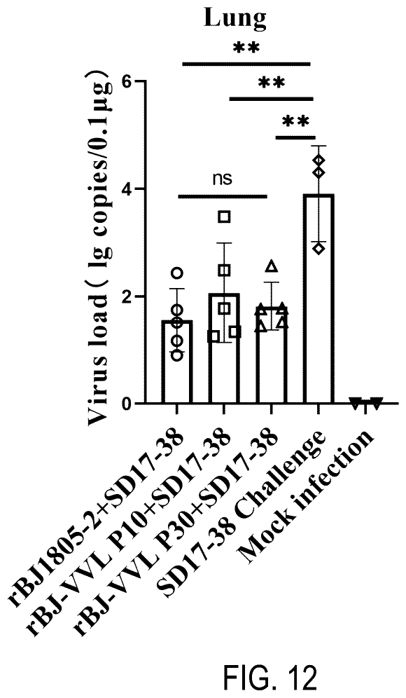

A viral load of the lung tissue was monitored by qPCR. Specific method and system for qPCR are described in Section 2. As shown in FIG. 12, the viral loads of lung tissue in the rBJ1805-2+SD17-38 group, rBJ-VVL P10+SD17-38 group and BJ-VVL P30+SD17-38 group were significantly lower than that of the SD17-38 challenge group. Moreover, no statistically significant differences in viral load were observed among the rBJ1805-2+SD17-38 group, the rBJ-VVL P10+SD17-38 group and the rBJ-VVL P30+SD17-38 group.

### 5. Pathological Anatomy, Pathological Sections and Immunohistochemistry

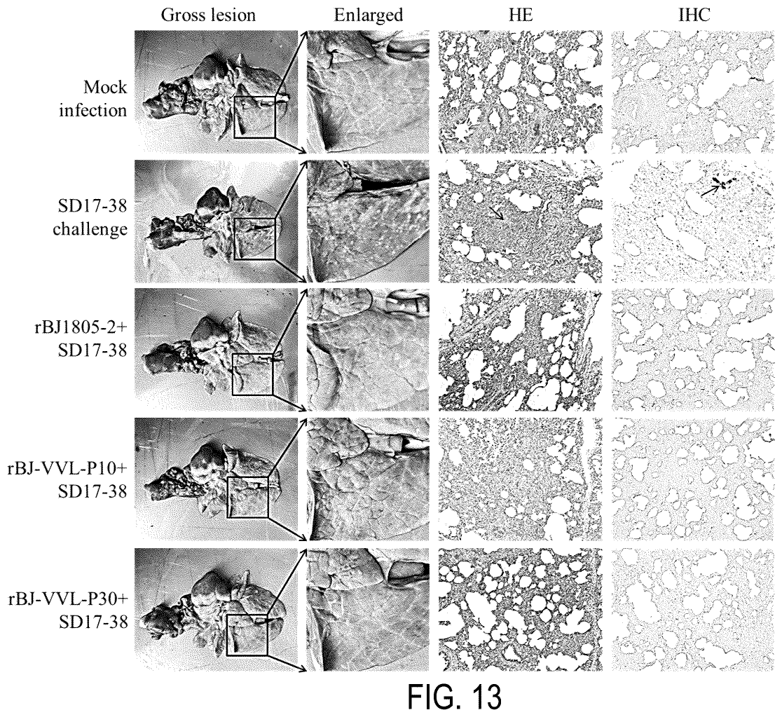

Results of piglet autopsy are shown in FIG. 13. The piglets in the SD17-38 group had obvious lung virus, a small amount of lymphocyte infiltration was observed by HE staining, and PRRSV antigen was detected by IHC. In contrast, the lungs of piglets in the rBJ1805-2+SD17-38 group, the rBJ-VVL P10+SD17-38 group, and the rBJ-VVL P30+SD17-38 group were similar to those in the negative control group, and exhibited no visible lung lesions. HE staining revealed no significant pathological changes, and no PRRSV antigen was detected by IHC. The above results indicated that immunization with rBJ1805-2, rBJ-VVL P10 and rBJ-VVL P30 could provide complete clinical protection for pigs.

What is claimed is:

1. An rBJ-VVL plasmid, wherein the rBJ-VVL plasmid is obtained by using a plasmid of a reverse genetics platform pACYC177-rBJ1805-2 as a template, designing a primer pair for amino acid mutations at positions 91, 97, and 98 of GP2a protein of an rBJ1805-2 virus to V, V and L, respectively, amplifying to obtain a mutated fragment by a PCR amplification method and then ligating the mutated fragment with a linearized vector; wherein a sequence of the plasmid of the reverse genetics platform pACYC177-rBJ1805-2 is shown in SEQ ID NO: 1+SEQ ID NO: 2; the rBJ1805-2 virus is an infectious clone virus of an NADC34-like PRRSV-2 rBJ1805-2 strain, which is deposited with China Center for Type Culture Collection, with a deposit address being Wuhan University, Wuhan, China, a deposit number being CCTCC NO: V202250 and a deposit date being Jun. 29, 2022; and an amino acid sequence of the GP2a protein of the rBJ1805-2 virus is amino acid sequence at positions 1888-2143 obtained by translating the sequence SEQ ID NO:2 at positions 2-9955.

2. The rBJ-VVL plasmid according to claim 1, wherein the primer pair comprises a combination of rBJ-XbaI-F3 and rBJ-VVL-R, and a combination of rBJ-VVL-F and rBJ-NOT1-2fu-1, which are amplified by the PCR amplification method to obtain front and rear segments of an rBJ-VVL mutant, respectively, and the rBJ-VVL plasmid is obtained by homologous recombination of the front and rear segments with the linearized vector; and sequences of the primer pair are shown in SEQ ID NO: 3, SEQ ID NO: 4, SEQ ID NO: 5, and SEQ ID NO: 6.

3. A mutant strain of NADC34-like PRRSV-2, wherein the mutant strain is obtained by mutating amino acids at positions 91, 97, and 98 of a GP2a protein of an infectious clone virus rBJ1805-2 of NADC34-like PRRSV-2 from T, M and F to V, V and L, respectively; the infectious clone virus rBJ1805-2 of the NADC34-like PRRSV-2 is deposited with China Center for Type Culture Collection, with a deposit address being Wuhan University, Wuhan, China, a deposit number being CCTCC NO: V202250 and a deposit date being Jun. 29, 2022; and an amino acid sequence of the GP2a protein of the rBJ1805-2 virus is amino acid sequence at positions 1888-2143 obtained by translating a sequence SEQ ID NO:2 at positions 2-9955.

4. The mutant strain according to claim 3, wherein the mutant strain is obtained by transfecting cells with the mutant plasmid in claim 1.

5. A method for constructing an rBJ-VVL plasmid, comprising the following steps:
(1) designing a mutant primer pair targeting amino acids at positions 91, 97, and 98 of a GP2a protein of an rBJ1805-2 virus; wherein the rBJ1805-2 virus is deposited with China Center for Type Culture Collection, with a deposit address being Wuhan University, Wuhan, China, a deposit number being CCTCC NO: V202250 and a deposit date being Jun. 29, 2022; and an amino acid sequence of the GP2a protein of the rBJ1805-2 virus is amino acid sequence at positions 1888-2143 obtained by translating a sequence SEQ ID NO: 2 at positions 2-9955; and sequences of the primer pair are shown in SEQ ID NO: 3, SEQ ID NO: 4, SEQ ID NO: 5, and SEQ ID NO: 6;
(2) using a plasmid of a reverse genetics platform pACYC177-rBJ1805-2 as a template, performing PCR amplification by using a combination of rBJ-XbaI-F3 and rBJ-VVL-R, and a combination of rBJ-VVL-F and rBJ-NOT1-2fu-1 to obtain front and rear segments of an rBJ-VVL mutant, and performing homologous recombination of a linearized vector fragment, the front fragment, and the rear fragment to obtain the rBJ-VVL plasmid, a sequence of the plasmid of the reverse genetics platform pACYC177-rBJ1805-2 is shown in SEQ ID NO:1+SEQ ID NO:2.

6. A method for obtaining a mutant strain or a passaged strain of NADC34-like PRRSV-2, comprising:
the steps (1) and (2) in claim 5, and a step (3) of transfecting cells with the rBJ-VVL plasmid; or performing further serial passage to obtain the passaged strain; and
the cells comprise PAM cells or Marc-145 cells.

7. Application of the rBJ-VVL plasmid in claim 1 in the preparation of a vaccine for preventing a porcine reproductive and respiratory syndrome virus.

8. An NADC34-like PRRSV-2-specific vaccine, wherein the NADC34-like PRRSV-2-specific vaccine comprises the mutant strain, a passaged strain thereof, or an inactivated strain thereof in claim 3.

9. The NADC34-like PRRSV-2-specific vaccine according to claim 8, wherein the vaccine is an injectable formulation, a drop formulation, or a spray formulation.

10. The mutant strain according to claim 3, wherein the mutant strain is obtained by transfecting cells with the mutant plasmid in claim 2.

11. Application of the rBJ-VVL plasmid in claim 2 in the preparation of a vaccine for preventing a porcine reproductive and respiratory syndrome virus.

12. Application of the mutant strain or a passaged strain thereof in claim 3 in the preparation of a vaccine for preventing a porcine reproductive and respiratory syndrome virus.

13. Application of the mutant strain or a passaged strain thereof in claim 4 in the preparation of a vaccine for preventing a porcine reproductive and respiratory syndrome virus.

14. An NADC34-like PRRSV-2-specific vaccine, wherein the NADC34-like PRRSV-2-specific vaccine comprises the mutant strain, a passaged strain thereof, or an inactivated strain thereof in claim 4.


SEQUENCE LISTING (39 sequences)

SEQ ID NO: 1 - DNA, 10057 bp, synthetic construct
  source 1..10057: mol_type = other DNA
gttgacgccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgcagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccttaataagatgatcttcttgagatcgttttggtctgcgcgtaatctcttgctctgaaaacgaaaaaaccgccttgcagggcggtttttcgaaggttctctgagctaccaactctttgaaccgaggtaactggcttggaggagcgcagtcaccaaaacttgtcctttcagtttagccttaaccggcgcatgacttcaagactaactcctctaaatcaattaccagtggctgctgccagtggtgcttttgcatgtctttccgggttggactcaagacgatagttaccggataaggcgcagcggtcggactgaacggggggttcgtgcatacagtccagcttggagcgaactgcctacccggaactgagtgtcaggcgtggaatgagacaaacgcggccataacagcggaatgacaccggtaaaccgaaaggcaggaacaggagagcgcacgagggagccgccagggggaaacgcctggtatctttatagtcctgtcgggtttcgccaccactgatttgagcgtcagatttcgtgatgcttgtcaggggggcggagcctatggaaaaacggctttgccgcggccctctcacttccctgttaagtatcttcctggcatcttccaggaaatctccgccccgttcgtaagccatttccgctcgccgcagtcgaacgaccgagcgtagcgagtcagtgagcgaggaagcggaatatatcctgtatcacatattctgctgacgcaccggtgcagccttttttctcctgccacatgaagcacttcactgacaccctcatcagtgccaacatagtaagccagtatacactccgctagcgctgaggtctgcctcgtgaagaaggtgttgctgactcataccaggcctgaatcgccccatcatccagccagaaagtgagggagccacggttgatgagagctttgttgtaggtggaccagttggtgattttgaacttttgctttgccacggaacggtctgcgttgtcgggaagatgcgtgatctgatccttcaactcagcaaaagttcgatttattcaacaaagccacgttgtgtctcaaaatctctgatgttacattgcacaagataaaaatatatcatcatgaacaataaaactgtctgcttacataaacagtaatacaaggggtgttatgagccatattcaacgggaaacgtcttgctcaaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctgacttgacgggacggcggctttgttgaataaatcgaacttttgctgagttgaaggatcagatcacgcatcttcccgacaacgcagaccgttccgtggcaaagcaaaagttcaaaatcaccaactggtccacctacaacaaagctctcatcaaccgtggctccctcactttctggctggatgatggggcgattcaggcctggtatgagtcagcaacaccttcttcacgaggcagacctcagcgctcaaagatgcaggggtaaaagctaaccgcatctttaccgacaaggcatccggcagttcaacagatcgggaagggctggatttgctgaggatgaaggtggaggaaggtgatgtcattctggtgaagaagctcgaccgtcttggccgcgacaccgccgacatgatccaactgataaaagagtttgatgctcagggtgtagcggttcggtttattgacgacgggatcagtaccgacggtgatatggggcaaatggtggtcaccatcctgtcggctgtggcacaggctgaacgccggaggatcccgatgtacgggccagatatacgcgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggactatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgttaattaatacatgaagtataggtgttggctctatgccaggacatttgtattgtcaggagctgtgaccactggcacagcccaaagcttgctgcacagaaacacccttctgtgacggcctccttcagggagtttaggggttgcccctagcaccttgcttctggagttgcactgctttacggtctctccaaccctttaaccatgtctgggattcttgatcggtgcacgtgcacccccaatgccagggtgtttatggcagagggccaagtctactgcacacgatgtctcagtgcacggtcccttctccccttgaacctccaagtctctgagcttggagtgttgggcttgttttataggcctgaagatccactccggtggacgttgccacgcgcattccccactgttgagtgctccccagctggggcttgttggctttccgcaattttcccaattgcacgaatgaccagtggaaacctgaacttccagcaaaggatggtacgggttgccgccgagatctacagagccggccaactcacctccacagttctgaagaacttgcaagtctatgaacggggttgccgttggtaccccatcgttggaccagttcctggggtttgcgtttacgccaactccctacatgtgagtgacaaacctttcccaggagctactcacgtactgaccaacctgccgctcccgcagagacctaaacctgaagatttttgcccctttgagtgtgccatggctgccgtctatgacatcggccatgatgccgtcatgttcgtggccgaggggagagtctcctgggctccacgtggtggggataagggaaggtttgaaattgctcccgaggggctaaggttgattgcagagcaactccatgcctcctttccgccccaccacatggtggacatgtcggaatttgccttcgtggcccccgagtgtggagcctccatgcgggtcgagcgccagtatggctgcctccccgctgacactgtccctgacggcaattgctggtggagcttgtttggctcgctcccattgaaagttcagtacgaagaaattcgccgtgctagccaatttggctatcagaccaagcacggcgtcgctggcaagtacctgcagcgaaggatgcaagtgaatggacttcgggcagtgactgacacgagtggacccatcgttgtacagtatttctgtgttaaggaaagctggattcgccacttgagattggcagaggaacctagcctcccggggtttgaggacctcatcagaataagagttgaacctaatacgtcgccactggttggtgaggacgagaaaatcttccggtttggcactcacaaatggtacggtgctggaaagagaacaagaaaagcgcgttctggtgcggccactaccggcactcgccgtgtctcacccgtccatgaaacccaacaagccaaagagcgcgaggacgtcagtgctaacaaggctgagcggctcgagcactattctccgcctgctgacgggaattgcggttggcactgtatttccgccatcgtcaaccatctggtgaattccagacttgaaaccacccttcccgagagggtaagaccatcggatgattgggctactgacgaaaatcttgttaatgccatccaaatcctcgggctccctgcgtccttagataggaacggtgcttgtgctggttccaggtacgtactcaagctagaaggcgagcactggactgtttccgtggcccctggggtgacccctcttctgctcccccttgaatgtgttcagggttgctgcgagcataagaacggtcttggtccttcagatgtggttgagttctccggactcaaccctacttgctctggccgactggcaggggtgatgcacttgcctagttgtgtcatcccagatgctttggctgaaatgcccgacgatcacagttgcccggcttctccaactgccactgtgtggaccacctcgcaattttatgctcgttacaggggaggagagcaccctgatcaggtgtgcttgaggaaaatcattagcctttgccaggtgcttgaggagtgctgttgttcccagagcaaaacccaccgggtcaccccggaagaggttgctgcaaagattgactcgtaccttcgcggtgctgcaaaccttgacgaatgcttggctaggcttgagaaagctcgcccgcctagcgtgatggacgcctcattaaattggaatgttgtgttccccggagtggaggcgggagttcacctaaccgagttgcccccggccgaccagtgtcgcgctcctacttctatcgcggctcgagggcttttggaggacggcccggtcccatcgccacctcttgcaccaagagttcagcctcgaaaaacaaggtccgttaaaagcttgccagattgcaagcccgtccctgctccgcgcaggagaatcagatctgactgtggcggtccgattttggtgggcgacaatgttcctaacggtcaggaagatttgactgttggcggtccatctgatcttccgacaccatctgagttaataacacctccgagtgagcccatgtttgtgcccgtatcgcgacacaactccaagccggttgcatctttgaacgggttagccccagttcctgtaccgcgcaaaactgtgtcccgacaaacggaacccttgagtgaatcaattcttgcgtccacacagcgatgcaaccttcagcaggtgaacggagcgaatctaacagccacaacgctgacgtgtcaagacgagcctctggatttatcttcatcctcactgatcggacatgaggcttccccttcaggactactacaaaatgtggacattccagggacaggagagcaagaagctgaagaggttctgaatggaatcccggacataccggacgacgctaaccctgcatctgtgtcatcaagcagctccttgtcgagtgtcgagattacgcgcccaaagtactcggctcaagccattattgactcaggtggaccctgttgtgggcacctccgagagttaaaagagaagtacctcagcgtgatgcgtgaggcatgtgatgttaccaagcttgatgaccctgtcacacaagagtggcttttccgtatgtgggacagggtagacatgctaacctggcgcaacacgtccatctttcaagcgcctttcactttggctgacaagttcaagtccctcccgaagatgatactcgagacaccgccaccctacccttgcgggttcgtaatgatgccacgcacacctgcaccttctgtggatgcagagagcgacatcaccgttggttcagttgccaccgaggatgttccgcacattctcggggaggtgcgaggtgtttgcgaaacgaccgtccaagaagtctcggcacccttcacggatgggccagcaagtgaccaacctgttggagagtcccaaacacaaacttctcccgcaggtgcagatggcgttggcttagttttggactctgaagaatcgtcaaagctcgtttacttgctgcctccaaacggcaccaacgctagtgaagggggaccgttacatgtggacaaaaagaaggctgaaaaatactttgaccaactgagccgacgggtttttggcatcgtttcccatctccctgtcttcttcgcacgtcttttcaggtctgacggtcattatgctccgggtgattggggttttgcagctttcactctattgtgcctctttctgtgttatagctacccggccttcggcgtggctcccctattgggtgtattttctgggtcttctcggcgtgtccgcatgggggtttttggttgttggttggctttcgctgttgggttgttcaaacctgtgcccgacccagtcggtactgcttgtgaatttgactcgccagagtgtagagacatccttcattcttttgagcttctgcaaccttgggaccctgttcgcagccttgtggtgggccccgtcggtctctgtcttgccattcttggcaggttattgggcggggcacgctacgtctggttgcttctgcttaggcttggcatcgtttcagactgtatcttggctggaacttatgtgctttcgcagggtaggtgtaaaaagtgttggggatcttgtgtaagaactgcccccagtgaggtcgccttcaatgttttcccttttacacgcgcaaccagatcgtcacttgtcgacctgtgtgatcggttttgtgcgcccaagggcgtggaccccatcttccttgccactggatggcgcgggtgctggtctggtcagagtcccattgagcaacccaccgagaaacctattgcgttcgcccagttggacgagaagaaaatcacggcaaggactgtagttgcccaaccttacgaccccaaccaagctgtgaagtgcttacgggtcttgcaggcgggtggggcgatggtggccgaggcagtcccaaaagtggtcaaagtctccgctgtcccatttcgagcccccttctttcccgccggcgtaaaagttgatcctgaatgcagggtcgtggtagacccggacacctttacagctgctcttcggtccggctactccaccacaaacctcattcttggtgtgggggattttgcccagttgaatgggttgaaaatcaggcagatctccaagtcctctgggggaggcccacacctcgcagctgccctgcatgttgcttgtgtgatggccctgcacatgcttgtcggaatttatgtgacctcggtgagcacctgcggcactggcactaacgacccgtggtgcaccaacccgtttgccgtccctggttatggacctggctccctctgcacggctaggttgtgcatctcccagcatggcctcacactacccttgacggcgctagtgaccggttttggcattcaggaaattgccttggttgttttgatcttcgtatccatcggtggcttggctcaccggttaagttgcaaggccgatgtgttgtgcgtcctgcttgcaatcgtcagctatgtttgggcgcctctcacttggttgatttgcgtgtttccttgctggttgcgctggttctccttgcatccccttaccattctatggttggtgtttttcttgatttccgtgaacatgccctcaggaatcttagctttagtattattgatttctctctggctcctcggtcgctacaccaatgttgccggccttgttaccccttatgacattcaccactataccaacggccctcgcggcgttgccgccttggccacagctcctgacgggacctatttggcagctgtccgccgcgctgcgttgactggccgtaccatgctgtttacaccgtctcaacttgggtcactccttgagggcgcccttagaacccaaaagccttcattgaacaccgtcaatgtggttgggtcctccatgggctccggcggggtgttcactatcgacgggaaaatcaaatgtgtaaccgccgcacacatccttacgggcaactctgccagggtctctggggctggtttcaaccagatgttggattttgatgtaaaaggggattttgccatagccgattgcccgggttggcaaggagttgctcccaaatcccagttttgcaaggatgggtggactggccgtgcttattggctgacgtcctctggcgtcgaacccggtgtcatcgggcagggatttgccttttgtttcaccgcgtgcggcgattctgggtccccagtgatcaccgaggccggggaacttgtcggtgtccacacgggatcgaataaacaaggaggaggcattgtcacgcgcccttcaggccagttttgtaatgtgacacccaccaaattaagtgaattgagtgaattcttcgctggacccaaggtcccgctcggtgatgtaaaagttggcaaccacataattaaagacacatacgaggtgccttctgatctttgtgctttgcttgctgctaaacctgagctggagggaggtctctctactgtccaacttctgtgtgtgttttttctcctgtggaggatgatgggacatacctggacgcctttggctgctgtggttttttttgtcttgaatgagatactcccagctgtcctggtccggagtgtcttctcctttggcatgtttgcgttgtcttggtttacaccatggtc

SEQ ID NO: 2 - DNA, 9955 bp, synthetic construct
  source 1..9955: mol_type = other DNA
cgcacaagtcctgatgatcaggcttctaacagctgctctcaacaggaacagggggtcactcgctttctacgtcctcggtgcagtgaccggatttgtcgcagatcttgcggtgactcaggggcatcctctacaggcagtggcgaacttaagcacctatgctttcctgcctcgaatgatggttgttacctcaccggttccattgcttgcttgtggcgttgtgcaccttcttgccataattttgtacctgttcaagcaccgttgcccgcattatatccttgttggcgatggagtgttttctagcgcctttttcttgcgatactttgctgaagggaagttgagggaaggggtgtcgcagtcctgtggaatgaatcacgagtcactgactggcgctctcgctatgagactcgatgacgaagacctggatttccttactaaactaactgattttaagtgctttgtttccgcatccaacatgaggaatgcggcgggccagtttatcgaggctgcttatgccaaagcacttagggtggaactttcccagttggtgcaagttgacaaggttcggggtgttttagctaagcttgaagcttttgctgataccgcgacaccccaactctcgcctggcgacattgtcgttgctcttggtcacacgcccgtcggcagcatctttgacttaaaagtaggcagcaccaagcacaccctacaagccatcgagactagagtcctcgctgggtccagaatgaccgtggcgcgcgttgttgatccgaatcccacacctccacccgtgcccgtgcccattcctctcccgccgaaagttttagagaacggcccccgtgcctgggatgatgaagaccgcctgaacaaaaaaaagcggcgcaaaatggaggccgttggcatttatgtcatggatgggaagaagtaccaaaaattctgggataagaattccggtgatgtgttctatgaagaagtccacgacaacacggacgcgtgggaatgccttagaaccgacgaccctgccgacttggatcctgagaaggggactttatgcggtcgcctcaccatagagaatagaccttaccatgtttatgcctccccttccggtaggaagtttcttgtccctgccaacccagagagtgggaaagcccagtgggaagctgctaggctttccatggagcaggcccttggcatgatgagcgtcgacggcgagttgaccgccaaggaagtggagaaattgaagagaataattgaaaaactccagggcctgactaaggagcagtgtttaaactgctagccgccagcggcttgacccgctgtggccgcggcggcttggttgttactgagacagcggtgaaaatagtcagatttcacagtcggaccttcaccctggggcctgtgaatttgaaagtggctagcgaggttgagctgaaagacgccgtcgagcacaaccaatacccaattgcaagaccagttgacggtggcgttgtgctcctgcgctctgcagtcccttcgcttatagacgtcctgatctccggggccgacgcatcccctcagtcactcgcccgtcatgggccgggaaacaccgggattgatggcacgctctgggattttgagtcagtagccactaaagaggaagtcgcactcagtgcacagataatacaggcttgtgacattaggcgcggtgatgctcctggaattggtctcccctacaagctgcatcctgtcaggggcaacccagaacgtgtgaagggggttttgaagaacacaaggtttggggacataccttacaagacccctagtgacactgggagcccagtacatgcggccgcctgtctcacgcctaatgccaccccggtgactgacgggcgctccgtcttggccaccaccatgccctctgggtttgagttgtatgtgccgaccattccagcgtctgtccttgattaccttgattccaggccagattgccctaaacagttgacggagcacgggtgtgaagatgctgcattgagagatctctccaaatatgacttgtccactcaaggttttgttttgcccggggtccttcgcctcgtacggaaatatttatttgcccacgtgggcaagtgcccgcctgtccaccggccctccacctacccagctaagaactccatggctggaataaacgggaataggttcccaaccaaggacattcagagcattcctgagatcgacgtcttgtgcgcacaggctgtacgagagaactggcagactgttaccccttgcactcttaagaaacagtattgcgggaagaagaaaaccaggaccatactcggcaccaataactttgtcgcgctggcccaccgggcagtgctgagcggtgtcacccagggcttcatgaaaaaggcattcaactcgcccatcgccctcgggaagaacaaattcaaggagctgcaaactccggtcctgggcaggtgccttgaggcagatcttgcatcttgcgatcgatccactcccgcgattgttcgctggtttgccgcccatctcctttatgaacttgcctgcgctgaagagtacctgtcgtcatatgtgctgaactgctgtcatgacctattggtcacgcaatccggtgcggtgactaagagaggtggcctatcatctggtgatccgatcacctcggtgtccaacaccatttacagtctggtcatttatgcgcagcacatggtactcagttatttcaaaagtggtcacccacatggcctcttgttccttcaggaccagctaaagtttgaggacatgctcaaggttcaacccctgattgtctattcggacgatcttgtgctgtatgccgagtctcccaccatgccaaactaccactggtgggttgagcacctgaatttgatgttagggtttcagacggacccgaagaaaacaaccatcactgactcgccgtctttcctgggctgcaggataatgaatgggtgccagttggtcccaaaccgagataggattctcgcagctcttgcttaccacatgaaagcgaataatgtttccgagtactacgcctccgccgctgcaatactcatggatagttgtgcttgtctagagtacgaccctgaatggtttgaagaacttgtggttgggatggcgcagtgcgcccgcaaggacggttacagtttccccggcccgccattcttcctatccatgtgggaaaaactcaggtctaattatgaggggaagaagtcaagggtgtgtgggtactgcggggcttcagccccatacgccactgcctgtggtcttgacgtctgtgtttaccacacccactttcaccagcattgtccagtcataatctggtgtggccacccagcgggttccgggtcctgtgatgagtgtaagcctcccacagggaaaggtacaagccctctggacgaggtcttaaaacaagttccatataagcctccacggactgtcctcatgcatgtggagcagggcctcaccccccttgacccaggcaggtatcagactcgtcgtgggttagttgccgttaggcgcgggattaggggaaatgaagtcgacctgccagacggtgactatgccagcaccgcgctactcccaacttgtaaagagatcaacatggtcgctgttgcttccaacgtactgcgtagtagatttatcatcggcccacccggtgctggaaaaacacattggctcctccaacaggttcaggatggtgatgtcatttacacaccgacccaccagaccatgcttgacatgattaaagctttggggacatgtcggtttaatgttccggcaggcacaacgctgcaatttcctgccccttcacgaacaggcccgtgggttcgcattctggccggcgggtggtgtcctggcaaaaattcattcctggacgaggctgcatactgcaaccaccttgatgtcctgaggctccttagtaaaactactctcacctgtttgggagacttcaaacaactccatccggtgggttttgattctcactgttatgtgtttgacgtcatgcctcaaactcaactgaagactatctggaggtttggacaaaatatctgtgatgccatccaaccagaatacagggacaagcttatgtctatggtcaacacaactcgtgtaacctatgtggaaaagcccgtcaaatatgggcaagtcctcaccccttaccatagggaccgcgaggatggcgccattaccattgattccagtcaaggtgccacatttgatgtggttacattgcatttgcccacgaaagattcactcagcaaacaaagggcccttgttgctatcaccagggcaagacatgccatctttgtgtatgacccacatgggcaactgcagagcttatttgatcttcctgcaaaaagcacgcccgttaacctggccgtgcactgtgatgggcaactgattgtgttagacagaaataacaaagaatgcacggttgcccaggctctaggtaatggtgacaaattcagagctacagacaagcgcgttgtggactccctccgcgccatttgtgccgacctggaggggtcgagctccccgctccccaaggttgcacataacttggggttttacttctcacctgatttgacacagttcgccaagcttccggcggaacttgcgccacattggccagtggtgacgacccaaaacaatgagaactggcctgatcgactggttgccagcctgcgccccattcacaaatatagctgtgcatgtgtcggtgccggctatatggtgggcccctcggtgtttttagggacccctggggtagtatcatactacctcacaaagtttgtgagaggcgaggctcaggtgcttccagaaacggtcttcagcactggccgaattgaggtggactgccgagagtatcttgacgatcgggagcgggaagttgcagcatctctcccgcacgcttttattggcgacgtcagaggcactactgtgggagggtgtcaccatgtcacttccaaatacctcccgcgcttcctccccaaggaatcagtcgcagtggtcggggtttcaagccccgggaaagccgcaaaggcggtgtgcacattgacagatgtgtacctcccagacctcgaggcttacctccatccagagaccctgtccaagtgctggaaattgatgttggactttaaagaagttcgactgatggtgtggaaggacaagacggcctatttccaactcgaaggtcgtcatttcacctggtatcagcttgctagctttgcttcgtacatccgtgttcctgtgaactccacggtgtacctggacccctgtatgggccccgccctttgcaacaggaaagttgtcgggtcccctcattggggagctgaccttgcagtcaccccttatgattacggagctagaaaaattttgtccagcgcataccatggcgagatgccccctgggtacaagattctggcgtgtgcagagttctcgctagacgacccagtcagatacaagcacacttgggggtttgaatcggatacagcgtacttgtacgagtatactggaaacggtgaggactgggaggattacaatgacgcgtttcgtgctcgacagaaagggagaatttacaaggccagtgctaccagcctgaagttccacttccctccgggtcacatcgttgaaccaacgttgggcctggactgaaatgaaatgggtgctttacaaagcctttttgataaaattggccaactttttgtggatgccttcacggagttcttggtgtctattgttgacattgtcatatttctggccatcttgtttggcttcacagtcgtcggttggctggttgtcttttgcatcagattggtttgctccacgatactccgtgcgcgccctgccgttcactctgagcagttacagaagatcctatgaggtcttccttcccctgtgccagacggatgtccccgtctggggaaccaagcaccccctgggaatgttttggcaccacaaggtgtcaaccctgattgatgaaatggtgtcgcgtagaatgtaccgcaccatggaaaatgcaggacaggctgcctggaaacaggtggtgagtgaggctactctgtctcgcatcagcagcttggatgtagtggctcatttccagcatctcgccgccattgaagccgagacctgtaaatatttggcctcccggctgcccatgctgcacaacctacgcatgacagggtcgaatgtaaccatagtgtataacagcactttgaatcgggtatctgctattttcccgacccccggctcccggccaaaacttcatgattttcagcaatggctaatagctgtacactcctctatattctcttctgttgcggcttcttgtactctctttgttgtgttgtggttgcgaattccaacgctacgttctgtttttggtttccgctggttaggggcaatttttccttcgagctccttgtgaattacacggtgtgccagccttgcctcactcgacaagcagccgctcagatttatgaacctggcagatccctttggtgcaggattgggcatgatcgatgtgagaagaacgaccatgatgagctaggatttgtggtaccgcctggtctttccagtgaaggccatttgaccagtgcttatgcctggttggcgttcttgtcctttagttatacagcccagtttcatcccgagatattcggaatcgggaatgtgagcaccgtttatgttgacaaacccaatcaactcatttgtgccgttcatgatgggcagaatgccaccttgtcccgctacgataacatcacagccgtgtaccagacctactatcaacatcaagttgatggcggcaattggtttcacctagaatggctgcgtcctttcttttcctcttggttggttttgaacgtttcatggtttctcaggcgtttgcctgtaaaccgtgtttcagttcgagtctttcagatatcaagaccaacactaccagcgcagcaagttccgttgtcctccaagacatcagctgtcttaggcataacaacccgccctctgcgacgtttcgcaaaatccctcagtgccgcacggcgataggaacacccgtttacatcactatcacagccaacgtgacggatgagaattatttgcattcctctgaccttctcatgctctcttcttgccttttctacgcttctgagatgagtgaaaagggatttaaggttatatttggcaatgtgtcaggcattgtggctgtatgtgtcaactttaccagctatgttcaacatatcaaggaggtcacccagcgatccttagtggttgaccatgtgcggctgcttcacttcatgacacctgaaactatgcggtgggcgaccgttttagcctgtctttttgccatcctgctggcgatttgaatgttcaggtatgttggggaaatgcttgaccgcgggctgctgctcgcaattgccttttttgtggtgtatcgtgccgttctgttttgttgcgctcgtcaacgccaacaacagcagcagctcccatttccagttgatttataacctgacgatatgtgagctgaatggcacagattggctaaataaaagttttgattgggcggttgagacctttgttatctttcctgtgttgactcatattgtctcctatggcgccctcaccaccagccactttcttgacacagtcggcctggccaccgtgtctgccgccggatatcaccacgggcggtatgtcttgagtagcatttacgccgtctgcgctttggctgcgttaatttgcttcgtcatcaggctaacaaaaaattgtatgtcctggcgttactcatgcaccagatacactaattttcttctggacaccaagggcaaactctatcgttggcggtctcctgtcatcatagagaaagggggtaaaattgaggtcgaaggtcacctcatcgaccttaagagagttgtgcttgacggttccgcggcaacccctgtaaccaaagtttcagcggaacaatggggtcgtccttagacgacttctgctatgacagcacagccccgcaaaaggtgcttttggcgttttccatcacctacacgccaataatgatatatgccttgaaggtaagtcgaggtcgactgctggggctcttgcacctcttaatattcctgaattgtgcctttacttttgggtatatgacatttattcattttgatagtacaaacaaggttgcacttaccatgggagcggtggttgctctcctctggggggtgtattcagccattgaaacctggagattcatcacctccagatgccggttgtgcttgctaggccgcaagtacattctggcccctgcccaccacgtcgaaagtgtcgcaggctttcatccgataacggcagatgataaccacgcatttgtcgtccggcgtcccggctccactacggtcaacggcacactggtgcccgggttgaaaagcctcgtgttgggtggcagaagagctgttaaacgaggagtggtgaaccttgttaaatatgccaaataacagcggcagacagcaaaataaaaagaagggggatggccagccagtcaatcagctatgccagatgttgggcaagattatcgcccaacagagccagtccagaggcaagggaccggggaagaagagtaagaataaaaacccggagaagccccattttcctctagcgactgaagatgacgtcagacatcactttacccccagtgagcgacaattgtgtctgtcgtcaatccggactgcctttaaccaaggcgctggaacttgcaccctgtcagattcagggagaataagttacactgtggagttcagtctgcctactcgtcacactgtgcgcttgattcgtgccacagcgtcaccttcagcatgatgagctggcattcttgggacatcccagtgcttgcattggaagaatgagcggtgaatggcactgattgacattgtgcctctaagtcacctattcaattagggcgaccgtatgggggtaatatttaattggcgagaaccatgcggccgaaattaaaaaaaaaaaaaaaaaaaaaggccggcatggtcccagcctcctcgctggcgccggctgggcaacattccgaggggaccgtcccctcggtaatggcgaatgggacggcgcgccctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggtagagcgcacgaatgagggccgacaggaagcaaagctgaaaggaatcaaatttggccgcaggcgtaccgtggacaggaacgtcgtgctgacgcttcatcagaagggcactggtgcaacggaaattgctcatcagctcagtattgcccgctccacggtttataaaattcttgaagacgaaagggcctcgtgatacgcctatttttataggttaatgtcatgataataatggtttcttagacgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgt

SEQ ID NO: 3 - DNA, 46 bp, synthetic construct
  source 1..46: mol_type = other DNA
cgtctggggagtcaagcaccccctgggagtgctttggcaccacaag

SEQ ID NO: 4 - DNA, 46 bp, synthetic construct
  source 1..46: mol_type = other DNA
cttgtggtgccaaagcactcccagggggtgcttgactccccagacg

SEQ ID NO: 5 - DNA, 31 bp, synthetic construct
  source 1..31: mol_type = other DNA
cgctgcaatactcatggatagttgtgcttgt

SEQ ID NO: 6 - DNA, 34 bp, synthetic construct
  source 1..34: mol_type = other DNA
caaacaacagatggctggcaactagaaggcacag

SEQ ID NO: 7 - DNA, 45 bp, synthetic construct
  source 1..45: mol_type = other DNA
caacgttgggcctggactgaaatgaaatggggtctatgcaaagcc

SEQ ID NO: 8 - DNA, 45 bp, synthetic construct
  source 1..45: mol_type = other DNA
ggctttgcatagaccccatttcatttcagtccaggcccaacgttg

SEQ ID NO: 9 - DNA, 38 bp, synthetic construct
  source 1..38: mol_type = other DNA
gccatcctactggcaatttgaatgttcaggtatgttgg

SEQ ID NO: 10 - DNA, 38 bp, synthetic construct
  source 1..38: mol_type = other DNA
ccaacatacctgaacattcaaattgccagtaggatggc

SEQ ID NO: 11 - DNA, 39 bp, synthetic construct
  source 1..39: mol_type = other DNA
ctcagtgccgcacggcgataggaacacccgtttacatca

SEQ ID NO: 12 - DNA, 39 bp, synthetic construct
  source 1..39: mol_type = other DNA
tgatgtaaacgggtgttcctatcgccgtgcggcactgag

SEQ ID NO: 13 - DNA, 36 bp, synthetic construct
  source 1..36: mol_type = other DNA
ggcaattggtttcacctggaatggctgcgtcctttc

SEQ ID NO: 14 - DNA, 36 bp, synthetic construct
  source 1..36: mol_type = other DNA
gaaaggacgcagccattccaggtgaaaccaattgcc

SEQ ID NO: 15 - DNA, 30 bp, synthetic construct
  source 1..30: mol_type = other DNA
gaactcatggtgaattacacggtgtgccag

SEQ ID NO: 16 - DNA, 30 bp, synthetic construct
  source 1..30: mol_type = other DNA
ctggcacaccgtgtaattcaccatgagttc

SEQ ID NO: 17 - DNA, 30 bp, synthetic construct
  source 1..30: mol_type = other DNA
cagcaatggctaatagctgtacattcctcc

SEQ ID NO: 18 - DNA, 30 bp, synthetic construct
  source 1..30: mol_type = other DNA
ggaggaatgtacagctattagccattgctg

SEQ ID NO: 19 - DNA, 28 bp, synthetic construct
  source 1..28: mol_type = other DNA
gcaattggtttcacctagaatggctacg

SEQ ID NO: 20 - DNA, 28 bp, synthetic construct
  source 1..28: mol_type = other DNA
cgtagccattctaggtgaaaccaattgc

SEQ ID NO: 21 - DNA, 29 bp, synthetic construct
  source 1..29: mol_type = other DNA
cagcaatggctaatagctgtacactcctc

SEQ ID NO: 22 - DNA, 29 bp, synthetic construct
  source 1..29: mol_type = other DNA
gaggagtgtacagctattagccattgctg

SEQ ID NO: 23 - DNA, 37 bp, synthetic construct
  source 1..37: mol_type = other DNA
caccctgagcaattacagaagatcttatgaggtcttc

SEQ ID NO: 24 - DNA, 37 bp, synthetic construct
  source 1..37: mol_type = other DNA
gaagacctcataagatcttctgtaattgctcagggtg

SEQ ID NO: 25 - DNA, 34 bp, synthetic construct
  source 1..34: mol_type = other DNA
ctgagcagttacagaagatcctatgaggcctttc

SEQ ID NO: 26 - DNA, 34 bp, synthetic construct
  source 1..34: mol_type = other DNA
gaaaggcctcataggatcttctgtaactgctcag

SEQ ID NO: 27 - DNA, 29 bp, synthetic construct
  source 1..29: mol_type = other DNA
gtcaaccctgattgatgaaatggtgtcgc

SEQ ID NO: 28 - DNA, 29 bp, synthetic construct
  source 1..29: mol_type = other DNA
gcgacaccatttcatcaatcagggttgac

SEQ ID NO: 29 - DNA, 37 bp, synthetic construct
  source 1..37: mol_type = other DNA
gtgtcaggtagatatccccacctggggaaccaagcac

SEQ ID NO: 30 - DNA, 37 bp, synthetic construct
  source 1..37: mol_type = other DNA
gtgcttggttccccaggtggggatatctacctgacac

SEQ ID NO: 31 - DNA, 46 bp, synthetic construct
  source 1..46: mol_type = other DNA
cgtctggggaaccaagcaccccctgggagtgctttggcaccacaag

SEQ ID NO: 32 - DNA, 46 bp, synthetic construct
  source 1..46: mol_type = other DNA
cttgtggtgccaaagcactcccagggggtgcttggttccccagacg

SEQ ID NO: 33 - DNA, 46 bp, synthetic construct
  source 1..46: mol_type = other DNA
cgtctggggagtcaagcaccccctgggaatgctttggcaccacaag

SEQ ID NO: 34 - DNA, 46 bp, synthetic construct
  source 1..46: mol_type = other DNA
cttgtggtgccaaagcattcccagggggtgcttgactccccagacg

SEQ ID NO: 35 - DNA, 46 bp, synthetic construct
  source 1..46: mol_type = other DNA
cgtctggggagtcaagcaccccctgggagtgttttggcaccacaag

SEQ ID NO: 36 - DNA, 46 bp, synthetic construct
  source 1..46: mol_type = other DNA
cttgtggtgccaaaacactcccagggggtgcttgactccccagacg

SEQ ID NO: 37 - DNA, 16 bp, synthetic construct
  source 1..16: mol_type = other DNA
tgtgagcaccgtttat

SEQ ID NO: 38 - DNA, 20 bp, synthetic construct
  source 1..20: mol_type = other DNA
tggttggcgttcttgtcctt

SEQ ID NO: 39 - DNA, 21 bp, synthetic construct
  source 1..21: mol_type = other DNA
catcatgaacggcacaaatga